(19) United States
(12) Patent Application Publication
(10) Pub. No.: US 2026/0265491 A1
(43) Pub. Date: Sep. 10, 2026

(54) AMINOINDOLE ANTIDEGRADANT COMPOUNDS AND USES THEREOF

(71) Applicant: FLEXSYS AMERICA L.P., Akron, OH (US)

(72) Inventors: Judicael Jacques CHAPELET, Copley, OH (US); Donald L. FIELDS, JR., Copley, OH (US); Frederick IGNATZ-HOOVER, Elyria, OH (US); Jonathan Michael PENNEY, Cuyahoga Falls, OH (US); Hiu Ching WONG, Tallmadge, OH (US)

(21) Appl. No.: 18/996,312

(22) PCT Filed: Jul. 21, 2023
(86) PCT No.: PCT/US2023/070705

§ 371 Date: Jan. 17, 2025

Related U.S. Application Data
(60) Provisional application No. 63/391,078, filed on Jul. 21, 2022.
(60) Provisional application No. 63/379,602, filed on Oct. 14, 2022.

(51) Int. Cl. C08K 5/3417; B60C 1/00; C08K 3/22; C08K 5/09; C08K 11/00

(52) U.S. Cl. CPC C08K 5/3417; B60C 1/00; C08K 3/22; C08K 5/09; C08K 11/00; C08K 2003/2234; C08K 2003/2237; C08K 2003/2296

(57) ABSTRACT

The present disclosure provides compositions comprising a compound represented by Formula (I): or salts or solvates thereof, wherein R¹, R^{2a}, R^{2b}, R^{2c}, R^{3a}, R^{3b}, and (II) are as defined in the specification. The present disclosure also provides vulcanized elastomeric articles, lubricant compositions, combustible fuel compositions, and fuel additive compositions comprising a compound or composition described herein. The present disclosure also provides processes for preparing the compositions and vulcanized elastomeric articles described herein. The present disclosure also provides processes for retreading tires using a compound or composition described herein. The present disclosure also provides kits comprising a compound or composition described herein.

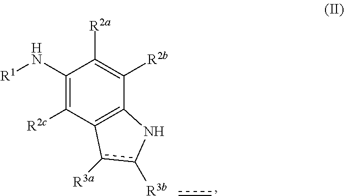

### BACKGROUND

### Field

The present disclosure provides compounds with antidegradant, e.g., antiozonant, antioxidant and/or antifatigue, properties that are useful additives for vulcanized rubber articles, compositions comprising elastomers, lubricants, fuels, and other compositions which require such properties or in compositions which are themselves useful as compositions to impart such properties.

### Background

Many materials such as plastics, elastomers, elastomeric products (tires, belts, hoses, bushings, mounts, vibration isolators, etc.), lubricants and petroleum products (such as hydraulic fluids, oils, fuels and oil/fuel additives for automotive and aviation applications) are prone to degradation upon prolonged exposure to light, heat, oxygen, ozone, repetitive mechanical actions and the like. Accordingly, compounds and compositions demonstrating antidegradant efficacy are well known in the art. For example, U.S. Pat. No. 8,987,515 discloses an aromatic polyamine useful in inhibiting oxidative degradation particularly in lubricant compositions. U.S. Patent Application Publication No. 2014/0316163 discloses antioxidant macromolecules with purported improved solubility in many commercially available oils and lubricants.

Antidegradants useful in the manufacture of articles formed from elastomers, plastics and the like require a very specific combination of qualities that can be difficult to achieve. While the antidegradants must obviously have commercially acceptable efficacy, they must also exhibit that efficacy over prolonged periods of time associated with use of the article, particularly at exposed surfaces of the article where degradation from environmental factors such as light, oxygen and ozone primarily occurs. Just as important to the protection of surface exposed components, efficacy in protecting imbedded components of composite materials from the effects of oxidative aging and repetitive mechanical action are critically important. The antidegradants must achieve these results while not negatively impacting other additives' efficacy or desirable characteristics in the final article. Further, antidegradants which provide or improve the mechanical fatigue life after an article has been in service, aged oxidatively or by exposure to ozone are highly valued since these will inherently improve the useful mechanical service life of article. Consequently, elastomeric articles which undergo repeated mechanical flexure, extension, or compression during service would greatly benefit from such a discovery.

Articles formed from general purpose elastomers such as natural rubber, in particular tires, are especially prone to degradation from both oxygen and ozone. As discussed in U.S. Pat. No. 2,905,654, the effect on rubber from degradation by oxygen is different from the effect from degradation from ozone; however, both effects can be detrimental to tire performance, appearance and life expectancy. Fatigue and crack propagation are also issues of specific concern, in particular for steel belt edge areas and tire sidewalls which are subject to significant stresses and stretching forces while flexed whether inflated, partially inflated, uninflated and throughout the service life of the tire. U.S. Pat. No. 8,833,417 describes an antioxidant system that purportedly increases long-term resistance to fatigue and crack propagation over the known antioxidants discussed immediately below.

Materials with antidegradant efficacy are well known in the art for use in tire applications and are commercially available. For example, N,N′-disubstituted-paraphenylenediamines such as those sold under the trademark Santoflex® are generally favored by many tire manufacturers for this purpose. EP 3147321 A1 discloses rubber compositions, tires, amine compounds, and anti-aging agents, and in particular, a rubber composition that is said to be suitable for use in tread rubber or sidewall rubber of a tire. U.S. Pat. No. 3,362,930 discloses 6-anilino-2,2,4-trimethyl-1,2-dihydroquinoline and its use as an antiozonant in a rubber composition.

### BRIEF SUMMARY

The present disclosure provides compounds that are useful as antidegradants, e.g., antiozonants, and/or antioxidants, and/or as additives in lubricants or combustible fuels, represented by any one of Formulae (I)-(VII) below, collectively referred to herein as “Compounds of the Disclosure” or individually as a “Compound of the Disclosure.”

The present disclosure also provides compositions comprising:
(i) a Compound of the Disclosure; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant, collectively referred to herein as “Compositions of the Disclosure” or individually as a “Composition of the Disclosure.”

The present disclosure also provides Compositions of the Disclosure comprising a Compound of the Disclosure and one or more elastomers.

The present disclosure also provides Compositions of the Disclosure comprising a Compound of the Disclosure and one or more fillers.

The present disclosure also provides Compositions of the Disclosure comprising a Compound of the Disclosure and one or more rubber chemicals.

The present disclosure also provides Compositions of the Disclosure comprising a Compound of the Disclosure and one or more plasticizers.

The present disclosure also provides Compositions of the Disclosure comprising a Compound of the Disclosure and a second antidegradant.

The present disclosure also provides Compositions of the Disclosure comprising a Compound of the Disclosure and one or more carriers.

The present disclosure also provides processes for preparing a composition comprising a Compound of the Disclosure and one or more carriers, the process comprising admixing the compound and the one or more carriers.

The present disclosure also provides vulcanized elastomeric articles comprising a Compound of the Disclosure.

The present disclosure also provides vulcanized elastomeric articles prepared using a composition described herein, e.g., Compositions of the Disclosure or compositions comprising a Compound of the Disclosure and one or more carriers.

The present disclosure also provides a process for preparing the vulcanized elastomeric articles described herein, the process comprising:
(a) forming a composition described herein into a formed shape; and
(b) vulcanizing the formed shape to provide a vulcanized elastomeric article.

The present disclosure also provides lubricant compositions comprising a lubricant and a Compound of the Disclosure.

The present disclosure also provides combustible fuel compositions comprising a combustible fuel and a Compound of the Disclosure.

The present disclosure also provides fuel additive compositions comprising a fuel additive and a Compound of the Disclosure.

The present disclosure also provides a process for retreading tires, the process comprising:
(a) applying a composition described herein to a tire;
(b) disposing a pre-vulcanized tread around the tire;
(c) disposing a curing envelope around the tire; and
(d) vulcanizing the tire.

The present disclosure also provides kits comprising a composition described herein and instructions for using the composition in a vulcanizable elastomeric composition.

The present disclosure also provides kits comprising a composition described herein and instructions for using the composition to prepare a vulcanized elastomeric article.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1 is a line graph depicting a ¹H nuclear magnetic resonance (NMR) spectrum of Compound 1 in d₆-dimethylsulfoxide (d₆-DMSO).

FIG. 2 is a line graph depicting a ¹³C NMR spectrum of Compound 1 in d₆-dimethylsulfoxide (d₆-DMSO).

FIGS. 3(a)-(c) are line graphs showing force retention as a function of time for sidewall compounds comprising Compound 1 under (a) static, (b) intermittent, and (c) dynamic conditions.

FIG. 4 is a line graph depicting a ¹H NMR spectrum of Compound 2 in d₆-dimethylsulfoxide (d₆-DMSO).

FIG. 5 is a line graph depicting a ¹³C NMR spectrum of Compound 2 in d₆-dimethylsulfoxide (d₆-DMSO).

FIG. 6 is a line graph depicting a ¹H NMR spectrum of Compound 19 in d₆-dimethylsulfoxide (d₆-DMSO).

FIG. 7 is a line graph depicting a ¹³C NMR spectrum of Compound 19 in d₆-dimethylsulfoxide (d₆-DMSO).

### DETAILED DESCRIPTION

In one embodiment, Compounds of the Disclosure are compounds having Formula (I):

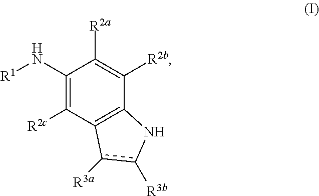

or a salt or solvate thereof, wherein:
[symbol] is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{2c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} and R^{3b} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl.

In some embodiments, Compounds of the Disclosure are compounds having Formula (I), wherein [symbol] is a double bond.

In some embodiments, Compounds of the Disclosure are compounds having Formula (I), wherein [symbol] is a single bond.

In some embodiments, Compounds of the Disclosure are compounds having Formula (I), wherein R^{3a} and R^{3b} are selected from the group consisting of hydrogen and C₁-C₆ alkyl.

In some embodiments, Compounds of the Disclosure are compounds having Formula (I), wherein R^{3a} and R^{3b} are hydrogen.

In some embodiments, Compounds of the Disclosure are compounds having Formula (II):

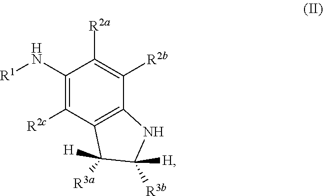

or a salt or solvate thereof, wherein:
R¹, R^{2a}, R^{2b}, R^{2c}, R^{3a}, and R^{3b} are defined in connection with Formula (I);
with the proviso that at least one of R^{3a} and R^{3b} is not hydrogen.

In some embodiments, Compounds of the Disclosure are compounds having Formula (III):

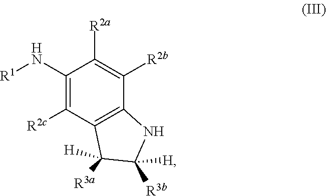

or a salt or solvate thereof, wherein:
R¹, R^{2a}, R^{2b}, R^{2c}, R^{3a}, and R^{3b} are defined in connection with Formula (I);
with the proviso that at least one of R^{3a} and R^{3b} is not hydrogen.

In some embodiments, Compounds of the Disclosure are compounds having Formula (IV):

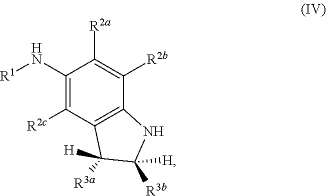

or a salt or solvate thereof, wherein:
R¹, R^{2a}, R^{2b}, R^{2c}, R^{3a}, and R^{3b} are defined in connection with Formula (I);
with the proviso that R^{3a} and R^{3b} are not hydrogen.

In some embodiments, Compounds of the Disclosure are compounds having Formula (V):

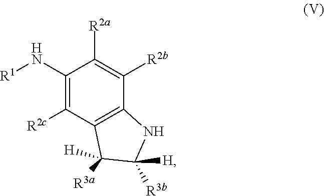

or a salt or solvate thereof, wherein:
R¹, R^{2a}, R^{2b}, R^{2c}, R^{3a}, and R^{3b} are defined in connection with Formula (I);
with the proviso that R^{3a} and R^{3b} are not hydrogen.

In some embodiments, Compounds of the Disclosure are compounds having Formula (VI):

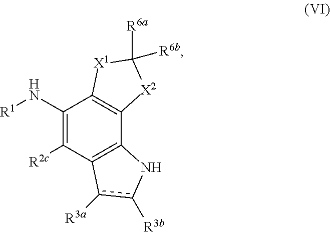

or a salt or solvate thereof, wherein:
X¹ is selected from the group consisting of —CR^{6c}R^{6d}—, —O—, —S—, and —NH—;
X² is selected from the group consisting of —(CR^{6e}R^{6f})ₚ—, —O—, —S—, and —NH—;
p is 1 or 2;
R^{6a} and R^{6b} are independently selected from the group consisting of hydrogen and C₁-C₆ alkyl; or
R^{6a} and R^{6b} taken together with the carbon atom to which they are attached form an oxo, i.e., —C(═O)—;
R^{6c} and R^{6d} are independently selected from the group consisting of hydrogen and C₁-C₆ alkyl;
each R^{6e} and R^{6f} are independently selected from the group consisting of hydrogen and C₁-C₆ alkyl; and
R¹, R^{2c}, R^{3a}, R^{3b}, and [symbol] are defined in connection with Formula (I).

In some embodiments, Compounds of the Disclosure are compounds having Formula (VII):

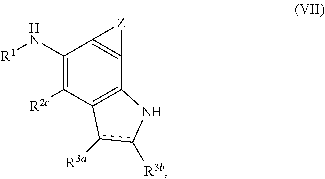

or a salt or solvate thereof, wherein:
Z is selected from the group consisting of —CR^{7a}═CR^{7b}—CR^{7c}═CR^{7d}—, —CR^{7a}═CR^{7b}—CHR^{7c}—CHR^{7d}—, —CHR^{7a}—CHR^{7b}—CHR^{7c}—CHR^{7d}—, —CR^{7a}═CR^{7b}—CHR^{7c}—, and —CHR^{7a}—CHR^{7b}—CHR^{7c}—;
R^{7a}, R^{7b}, R^{7c}, and R^{7d} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R¹, R^{2c}, R^{3a}, R^{3b}, R⁴, R⁵, and [symbol] are defined in connection with Formula (I).

In some embodiments, Compounds of the Disclosure are compounds having Formula (VII), wherein Z is —CR^{7a}═CR^{7b}—CR^{7c}═CR^{7d}—. In some embodiments, R^{7a}, R^{7b}, R^{7c}, and R^{7d} are hydrogen.

In some embodiments, Compounds of the Disclosure are compounds having Formula (VII), wherein Z is —CR^{7a}═CR^{7b}—CHR^{7c}—CHR^{7d}—. In some embodiments, R^{7a}, R^{7b}, R^{7c}, and R^{7d} are hydrogen.

In some embodiments, Compounds of the Disclosure are compounds having Formula (VII), wherein Z is —CHR^{7a}—CHR^{7b}—CHR^{7c}—CHR^{7d}—. In some embodiments, R^{7a}, R^{7b}, R^{7c}, and R^{7d} are hydrogen.

In some embodiments, Compounds of the Disclosure are compounds having Formula (VII), wherein Z is —CR^{7a}═CR^{7b}—CHR^{7c}—. In some embodiments, R^{7a}, R^{7b}, and R^{7c} are hydrogen.

In some embodiments, Compounds of the Disclosure are compounds having Formula (VII), wherein Z is —CHR^{7a}—CHR^{7b}—CHR^{7c}—. In some embodiments, R^{7a}, R^{7b}, and R^{7c} are hydrogen.

In some embodiments, Compounds of the Disclosure are compounds having Formula (VII), wherein [symbol] is a double bond.

In some embodiments, Compounds of the Disclosure are compounds having Formula (VII), wherein [symbol] is a single bond.

In some embodiments, Compounds of the Disclosure are compounds of Formula (I), wherein R¹ is selected from the group consisting of —CHR^{1a}R^{1b}, optionally substituted 4- to 6-membered heterocyclyl, and optionally substituted 5- or 6-membered heteroaryl.

In some embodiments, Compounds of the Disclosure are compounds of any one of Formulae (I)-(VII), wherein R¹ is selected from the group consisting of C₁-C₁₂ alkyl and optionally substituted phenyl.

In some embodiments, Compounds of the Disclosure are compounds of any one of Formulae (I)-(VII), wherein R¹ is phenyl.

In some embodiments, Compounds of the Disclosure are compounds of any one of Formulae (I)-(VII), wherein R¹ is selected from the group consisting of methyl, ethyl, propyl, isopropyl, tert-butyl, sec-butyl, iso-butyl, pentyl, hexyl, heptyl, octyl, nonyl, decyl, undecyl, and dodecyl.

In some embodiments, Compounds of the Disclosure are compounds of any one of Formulae (I)-(VII), wherein R¹ is sec-butyl.

In some embodiments, Compounds of the Disclosure are compounds of any one of Formulae (I)-(VII), wherein R¹ is isopropyl.

In some embodiments, Compounds of the Disclosure are compounds of any one of Formulae (I)-(VII), wherein R¹ is —CHR^{1a}R^{1b}.

In some embodiments, Compounds of the Disclosure are compounds of Formula (I), wherein R^{1a} is selected from the group consisting of optionally substituted 4- to 6-membered heterocyclyl and optionally substituted 5- or 6-membered heteroaryl.

In some embodiments, Compounds of the Disclosure are compounds of any one of Formulae (I)-(VII), wherein R^{1a} is selected from the group consisting of

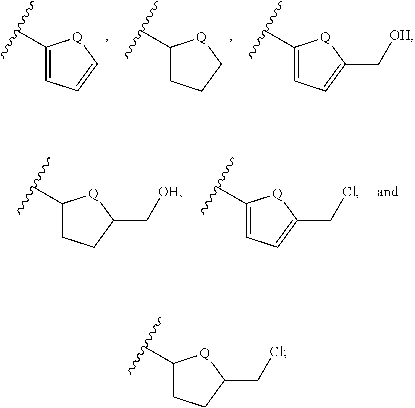

and
Q is selected from the group consisting of —NH—, —O—, and —S—.

In some embodiments, Compounds of the Disclosure are compounds of any one of Formulae (I)-(VII), wherein Q is —O—.

In some embodiments, Compounds of the Disclosure are compounds of any one of Formulae (I)-(VII), wherein R^{1a} is selected from the group consisting of:

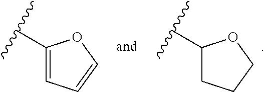 [SMILES: *c1ccco1 ; *C1CCCO1]

In some embodiments, Compounds of the Disclosure are compounds of any one of Formulae (I)-(VII), wherein R^{1b} is selected from the group consisting of hydrogen and methyl.

In some embodiments, Compounds of the Disclosure are compounds of any one of Formulae (I)-(V), wherein R^{2a}, R^{2b}, and R^{2c} are independently selected from the group consisting of hydrogen, halogen, C₁-C₉ alkyl, C₁-C₆ alkoxy, and C₁-C₆ alkylthio.

In some embodiments, Compounds of the Disclosure are compounds of any one of Formulae (I)-(V), wherein R^{2a} is selected from the group consisting of C₁-C₉ alkyl, C₁-C₆ alkoxy, and C₁-C₆ alkylthio; and R^{2b} and R^{2c} are hydrogen.

In some embodiments, Compounds of the Disclosure are compounds of any one of Formulae (I)-(V), wherein R^{2b} is selected from the group consisting of C₁-C₉ alkyl, C₁-C₆ alkoxy, and C₁-C₆ alkylthio; and R^{2a} and R^{2c} are hydrogen.

In some embodiments, Compounds of the Disclosure are compounds of any one of Formulae (I)-(V), wherein R^{2c} is selected from the group consisting of C₁-C₉ alkyl, C₁-C₆ alkoxy, and C₁-C₆ alkylthio; and R^{2a} and R^{2b} are hydrogen.

In some embodiments, Compounds of the Disclosure are compounds of any one of Formulae (I)-(V), wherein R^{2a}, R^{2b}, and R^{2c} are hydrogen.

In some embodiments, Compounds of the Disclosure are compounds of Formula (I), wherein:
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl.

In some embodiments, Compounds of the Disclosure are compounds of Formula (I), with the proviso that when R¹ is:

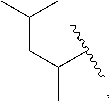 [SMILES: *C(C)CC(C)C]

R^{2a}, R^{2b}, and R^{2c} are hydrogen, R^{3a} is hydrogen, and [symbol] is a double bond, then R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl.

In some embodiments, Compounds of the Disclosure are compounds of Formula (I), with the proviso that the compound is not:

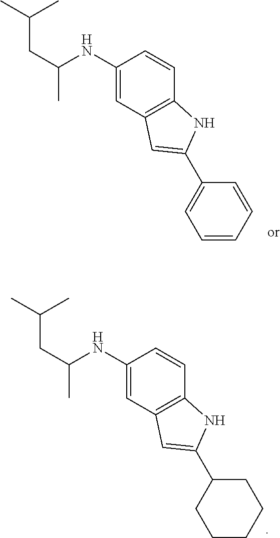 [SMILES: CC(C)CC(C)Nc1ccc2[nH]c(-c3ccccc3)cc2c1 ; CC(C)CC(C)Nc1ccc2[nH]c(C3CCCCC3)cc2c1]

In some embodiments, Compounds of the Disclosure are any one or more of the compounds of Table 1, or a salt or solvate thereof.

TABLE 1

| Compound No. | Structure |
|---|---|
| 1 | 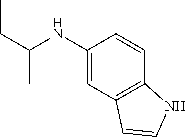 |
| 2 | 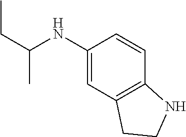 |
| 3 | 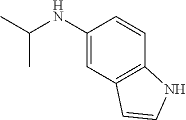 |
| 4 | 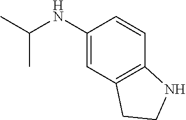 |
| 5 | 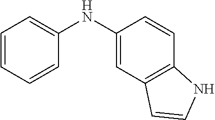 [SMILES: c1ccc(Nc2ccc3[nH]ccc3c2)cc1] |
| 6 | 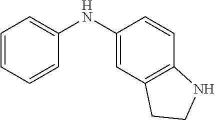 [SMILES: c1ccc(Nc2ccc3c(c2)CCN3)cc1] |
| 7 | 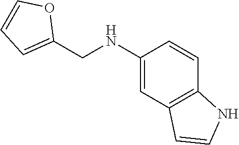 [SMILES: c1coc(CNc2ccc3[nH]ccc3c2)c1] |
| 8 | 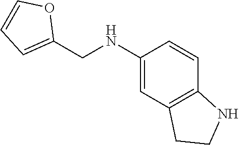 [SMILES: c1coc(CNc2ccc3c(c2)CCN3)c1] |
| 9 | 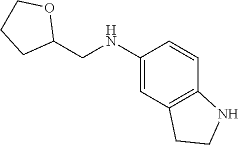 [SMILES: c1cc2c(cc1NCC1CCCO1)CCN2] |
| 10 | 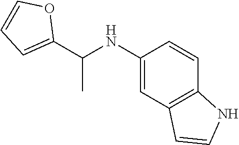 [SMILES: CC(Nc1ccc2[nH]ccc2c1)c1ccco1] |
| 11 | 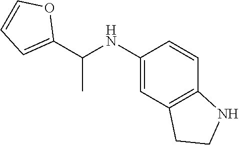 [SMILES: CC(Nc1ccc2c(c1)CCN2)c1ccco1] |
| 12 | 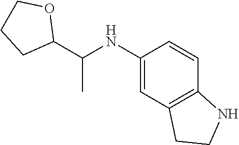 [SMILES: CC(Nc1ccc2c(c1)CCN2)C1CCCO1] |
| 13 | 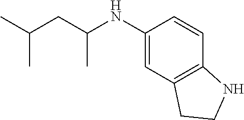 [SMILES: CC(C)CC(C)Nc1ccc2c(c1)CCN2] |
| 14 | 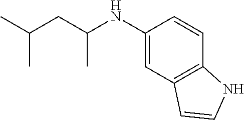 [SMILES: CC(C)CC(C)Nc1ccc2[nH]ccc2c1] |
| 15 | 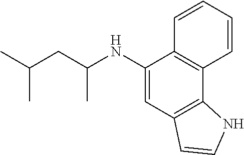 [SMILES: CC(C)CC(C)Nc1cc2cc[nH]c2c2ccccc12] |
| 16 | 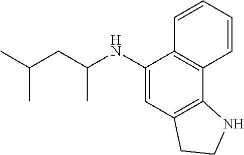 [SMILES: CC(C)CC(C)Nc1cc2c(c3ccccc13)NCC2] |
| 17 | 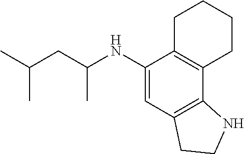 [SMILES: CC(C)CC(C)Nc1cc2c(c3c1CCCC3)NCC2] |
| 18 | 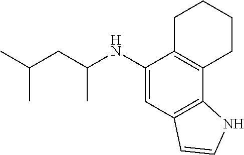 [SMILES: CC(C)CC(C)Nc1cc2cc[nH]c2c2c1CCCC2] |
| 19 | 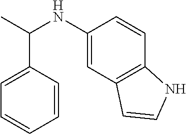 [SMILES: CC(Nc1ccc2[nH]ccc2c1)c1ccccc1] |
| 20 | 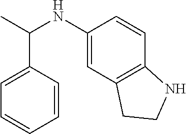 [SMILES: CC(Nc1ccc2c(c1)CCN2)c1ccccc1] |
| 21 | 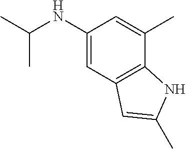 |
| 22 | 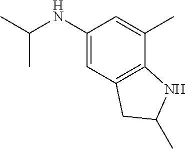 |
| 23 | 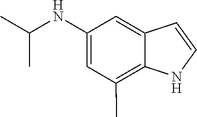 |
| 24 | 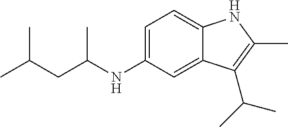 [SMILES: Cc1[nH]c2ccc(NC(C)CC(C)C)cc2c1C(C)C] |
| 25 | 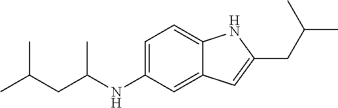 [SMILES: CC(C)Cc1cc2cc(NC(C)CC(C)C)ccc2[nH]1] |
| 26 | 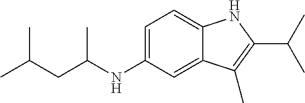 [SMILES: Cc1c(C(C)C)[nH]c2ccc(NC(C)CC(C)C)cc12] |
| 27 | 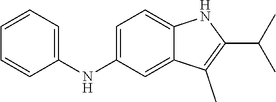 [SMILES: Cc1c(C(C)C)[nH]c2ccc(Nc3ccccc3)cc12] |
| 28 | 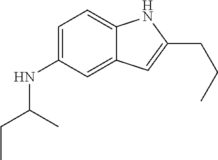 [SMILES: CCCc1cc2cc(NC(C)CC)ccc2[nH]1] |
| 29 | 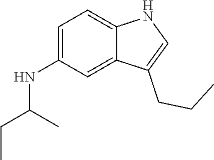 [SMILES: CCCc1c[nH]c2ccc(NC(C)CC)cc12] |
| 30 | 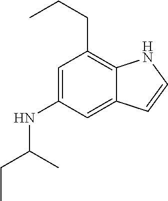 [SMILES: CCCc1cc(NC(C)CC)cc2cc[nH]c12] |
| 31 | 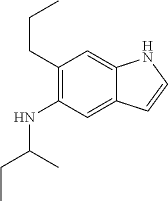 [SMILES: CCCc1cc2[nH]ccc2cc1NC(C)CC] |
| 32 | 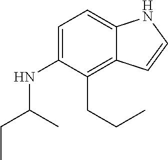 [SMILES: CCCc1c(NC(C)CC)ccc2[nH]ccc12] |
| 33 | 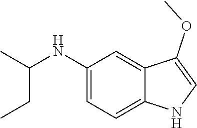 [SMILES: CCC(C)Nc1ccc2[nH]cc(OC)c2c1] |

Compounds of the Disclosure can be useful as antidegradants, e.g., antiozonants and/or antioxidants, as additives in lubricants, and as additives in combustible fuels.

Compounds of the Disclosure can be prepared by reacting 5-aminoindole or 5-aminoindoline with an aldehyde or ketone via reductive alkylation, as shown in Scheme 1.

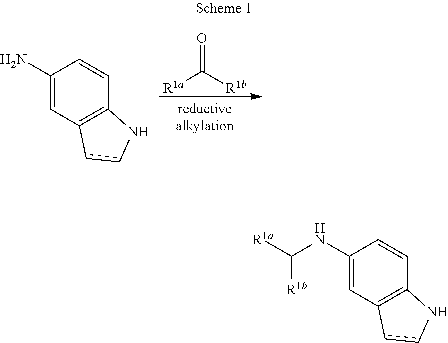

### Definitions

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one embodiment the alkyl is a straight-chain alkyl. In another embodiment, the alkyl is a branched-chain alkyl. In one embodiment, the alkyl is a C₁-C₈ alkyl. In another embodiment, the alkyl is a C₁-C₆ alkyl. In another embodiment, the alkyl is a C₁-C₄ alkyl. In another embodiment, the alkyl is a C₁-C₃ alkyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “halo” or “halogen” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “amino” as used by itself or as part of another group refers to a radical of the formula —NR^{1f}R^{1g}, wherein R^{1f} and R^{1g} are independently hydrogen or alkyl.

In one embodiment, the amino is —NH₂.

In another embodiment, the amino is an “alkylamino,” i.e., an amino group wherein R¹ is C₁₋₆ alkyl and R^{1g} is hydrogen. In one embodiment, R^{1f} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{1f} and R^{1g} are each independently C₁₋₆ alkyl. In one embodiment, R^{1f} and R^{1g} are each independently C₁-C₄ alkyl. Non-limiting exemplary dialkylamino groups include —N(CH₃)₂ and —N(CH₃)CH₂CH(CH₃)₂.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “haloalkyl” as used herein by itself or as part of another group refers to an alkyl substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl and the resulting haloalkyl is referred to as a “C₁-C₆ haloalkyl.” In another embodiment, the alkyl is a C₁-C₄ alkyl and the resulting haloalkyl is referred to as a “C₁-C₄ haloalkyl.” In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The term “alkoxy” as used herein by itself or as part of another group refers to an alkyl attached to a terminal oxygen atom. In one embodiment, the alkyl is a C₁-C₆ alkyl, and the resulting alkoxy is referred to as a “C₁-C₆ alkoxy.” In another embodiment, the alkyl is a C₁-C₄ alkyl group and thus the resulting alkoxy is referred to as a “C₁-C₄ alkoxy.” Non-limiting exemplary alkoxy groups include methoxy, ethoxy, and tert-butoxy.

The term “alkylthio” as used herein by itself or as part of another group refers to an alkyl group attached to a terminal sulfur atom. In one embodiment, the alkyl is a C₁-C₆ alkyl, and the resulting alkylthio is referred to as a “C₁-C₆ alkylthio.” In one embodiment, the alkyl group is a C₁-C₄ alkyl group and the resulting alkylthio is referred to as a “C₁-C₄ alkylthio.” Non-limiting exemplary alkylthio groups include —SCH₃, and —SCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃-C₁₂ cycloalkyl, or the number of carbons designated, e.g., a C₃-C₆ cycloalkyl such a cyclopropyl, cyclobutyl, cyclopentyl, and cyclohexyl. In one embodiment, the cycloalkyl is bicyclic, i.e., it has two rings. In another embodiment, the cycloalkyl is monocyclic, i.e., it has one ring. In another embodiment, the cycloalkyl is a C₃-C₈ cycloalkyl. In another embodiment, the cycloalkyl is a C₃-C₆ cycloalkyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃-C₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl that is either unsubstituted or substituted with one to four substituents, wherein the substituents are independently halo, nitro, cyano, hydroxyl, amino, (e.g., —NH₂, alkylamino, or dialkylamino), alkoxy, —SH, alkylthio, alkylcarbonyl, —C(═O)OR⁸, —OC(═O)R⁸, —C(═O)NR⁸R⁹, —NR⁸C(═O)R⁹, —SC(═O)R⁸, —SC(═O)SR⁸, —SC(═O)NR⁸R⁹, —NR⁸C(═O)SR⁹, —NR⁸C(═S)SR⁹, alkyl, haloalkyl, optionally substituted cycloalkyl, optionally substituted heterocyclyl, optionally substituted aryl, or optionally substituted heteroaryl, wherein:
R⁸ is hydrogen or C₁-C₉ alkyl; and
R⁹ is hydrogen or C₁-C₉ alkyl.

The term “aryl” as used herein by itself or as part of another group refers to aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to an aryl that is either unsubstituted or substituted with one to four substituents, wherein the substituents are independently halo, nitro, cyano, hydroxyl, amino, (e.g., —NH₂, alkylamino, or dialkylamino), alkoxy, —SH, alkylthio, alkylcarbonyl, —C(═O)OR⁸, —OC(═O)R⁸, —C(═O)NR⁸R⁹, —NR⁸C(═O)R⁹, —SC(═O)R⁸, —SC(═O)SR⁸, —SC(═O)NR⁸R⁹, —NR⁸C(═O)SR⁹, —NR⁸C(═S)SR⁹, alkyl, haloalkyl, optionally substituted cycloalkyl, optionally substituted heterocyclyl, optionally substituted aryl, or optionally substituted heteroaryl, wherein R⁸ and R⁹ are as defined in connection with the term “optionally substituted cycloalkyl.”

The term “optionally substituted phenyl” as used herein by itself or as part of another group refers to phenyl that is either unsubstituted or substituted with one to five substitutents, wherein the substituents are each independently halo, nitro, cyano, hydroxyl, amino, (e.g., —NH₂, alkylamino, or dialkylamino), alkoxy, —SH, alkylthio, alkylcarbonyl, —C(═O)OR⁸, —OC(═O)R⁸, —C(═O)NR⁸R⁹, —NR⁸C(═O)R⁹, —SC(═O)R⁸, —SC(═O)SR⁸, —SC(═O)NR⁸R⁹, —NR⁸C(═O)SR⁹, —NR⁸C(═S)SR⁹, alkyl, haloalkyl, optionally substituted cycloalkyl, optionally substituted heterocyclyl, optionally substituted aryl, or optionally substituted heteroaryl, wherein R⁸ and R⁹ are as defined in connection with the term “optionally substituted cycloalkyl.”

The term “heterocyclyl” or “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18-membered heterocyclyl, comprising one, two, three, or four heteroatoms. Each heteroatom is independently oxygen, sulfur, or nitrogen. Each sulfur atom may be independently oxidized to give a sulfoxide, i.e., S(═O), or sulfone, i.e., S(═O)₂. The term heterocyclyl includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclyl also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one. In some embodiments, heterocyclyl is a 6-membered ring comprising one nitrogen atom. The heterocyclyl may be fused to the rest of the molecule to form a bicyclic group, e.g., 1,2-dihydroquinoline or 1,2,3,4-tetrahydroquinoline.

The term “optionally substituted heterocyclyl” as used herein by itself or part of another group refers to a heterocyclyl group that is either unsubstituted or substituted with one to four substituents, independently halo, nitro, cyano, hydroxyl, amino, (e.g., —NH₂, alkylamino, or dialkylamino), alkoxy, —SH, alkylthio, alkylcarbonyl, —C(═O)OR⁸, —OC(═O)R⁸, —C(═O)NR⁸R⁹, —NR⁸C(═O)R⁹, —SC(═O)R⁸, —SC(═O)SR⁸, —SC(═O)NR⁸R⁹, —NR⁸C(═O)SR⁹, —NR⁸C(═S)SR⁹, alkyl, haloalkyl, optionally substituted cycloalkyl, optionally substituted heterocyclyl, optionally substituted aryl, or optionally substituted heteroaryl, wherein R⁸ and R⁹ are as defined in connection with the term “optionally substituted cycloalkyl.”

The term “heteroaryl” as used herein by itself or as part of another group refers to monocyclic aromatic ring systems having five to six ring members, i.e., a 5- to 6-membered heteroaryl, comprising one, two, three, four, or five heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl has 5 ring atoms, e.g., furyl, a 5-membered heteroaryl having four carbon atoms and one oxygen atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, furyl, pyranyl, 2H-pyrrolyl, pyrrolyl, imidazolyl, pyrazolyl, pyridyl, pyrazinyl, pyrimidinyl, pyridazinyl, thiazolyl, isothiazolyl, and isoxazolyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxyl, amino, (e.g., —NH₂, alkylamino, or dialkylamino), alkoxy, —SH, alkylthio, alkylcarbonyl, —C(═O)OR⁸, —OC(═O)R⁸, —C(═O)NR⁸R⁹, —NR⁸C(═O)R⁹, —SC(═O)R⁸, —SC(═O)SR⁸, —SC(═O)NR⁸R⁹, —NR⁸C(═O)SR⁹, —NR⁸C(═S)SR⁹, alkyl, haloalkyl, optionally substituted cycloalkyl, optionally substituted heterocyclyl, optionally substituted aryl, or optionally substituted heteroaryl, wherein R⁸ and R⁹ are as defined in connection with the term “optionally substituted cycloalkyl.”

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

In thin layer chromatography (TLC), the term R_{f} stands for retention factor. R_{f} is defined as the distance travelled by an individual component divided by the total distance travelled by the eluent. Its value is always between zero and one.

Salts and solvates, e.g., hydrates, of the Compounds of the Disclosure can also be used in the methods disclosed herein.

The present disclosure encompasses the preparation and use of salts of Compounds of the Disclosure. Salts of Compounds of the Disclosure can be prepared during the final isolation and purification of the compounds or separately by reacting the compound with an acid having a suitable cation. Salts of Compounds of the Disclosure can be acid addition salts formed with acceptable acids. Examples of acids which can be employed to form salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerophosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. In light of the foregoing, any reference to Compounds of the Disclosure appearing herein is intended to include Compounds of the Disclosure as well as salts, hydrates, or solvates thereof.

The present disclosure encompasses the preparation and use of solvates of Compounds of the Disclosure. The term “solvate” as used herein is a combination, physical association and/or solvation of a compound of the present disclosure with a solvent molecule such as, e.g., a desolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a solvent, such as water, methanol, and ethanol, and it is intended that the disclosure includes both solvated and unsolvated forms of Compounds of the Disclosure.

One type of solvate is a hydrate. A “hydrate” relates to a particular subgroup of solvates where the solvent molecule is water. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “wt/wt %” as used herein refers to the mass of one component in a composition or blend, e.g., a composition comprising a Compound of the Disclosure and one or more elastomers; or a Composition of the Disclosure and one or more fillers; or a blend comprising two or more elastomers, etc., divided by the combined mass of all components in the composition or blend, times 100. For example, the wt/wt % of a Compound of the Disclosure in a composition comprising 1 kg of the compound, 1 kg natural rubber, and 2 kg of synthetic rubber, is 25 wt/wt % (1 kg/4 kg=0.25×100=25 wt/wt %). The wt/wt % of a Compound of the Disclosure in a composition comprising 1 kg of the compound and 1 kg of carbon black is 50 wt/wt % (1 kg/2 kg=0.20×100=50 wt/wt %). The wt/wt % of a Compound of the Disclosure in a composition comprising 1 kg of the compound, 1 kg natural rubber, 2 kg of synthetic rubber, and 1 kg of carbon black is 20 wt/wt % (1 kg/5 kg=0.20×100=20 wt/wt %). The wt/wt % of natural rubber in a blend of one or more elastomers comprising 20 kg natural rubber and 30 kg synthetic rubber is 40 wt/wt % (20 kg/50 kg=0.40×100=40 wt/wt %).

### Compositions and Methods of Use

In another embodiment, the disclosure provides compositions comprising:
(i) a Compound of the Disclosure; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant.

In some embodiments, a Composition of the Disclosure comprises from about 15 wt/wt % to about 85 wt/wt % of a Compound of the Disclosure. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of a Compound of the Disclosure. In some embodiments, the composition comprises from about 0.1 wt/wt % to about 0.5 wt/wt %, from about 0.1 wt/wt % to about 1 wt/wt %, from about 0.1 wt/wt % to about 1.5 wt/wt %, from about 0.1 wt/wt % to about 2 wt/wt %, from about 0.1 wt/wt % to about 2.5 wt/wt %, from about 0.1 wt/wt % to about 3 wt/wt %, from about 0.1 wt/wt % to about 3.5 wt/wt %, from about 0.1 wt/wt % to about 4 wt/wt %, from about 0.1 wt/wt % to about 4.5 wt/wt %, from about 0.1 wt/wt % to about 5 wt/wt %, from about 0.1 wt/wt % to about 6 wt/wt %, from about 0.1 wt/wt % to about 7 wt/wt %, from about 0.1 wt/wt % to about 8 wt/wt %, from about 0.1 wt/wt % to about 9 wt/wt %, from about 0.1 wt/wt % to about 10 wt/wt %, from about 0.5 wt/wt % to about 1 wt/wt %, from about 0.5 wt/wt % to about 1.5 wt/wt %, from about 0.5 wt/wt % to about 2 wt/wt %, from about 0.5 wt/wt % to about 2.5 wt/wt %, from about 0.5 wt/wt % to about 3 wt/wt %, from about 0.5 wt/wt % to about 3.5 wt/wt %, from about 0.5 wt/wt % to about 4 wt/wt %, from about 0.5 wt/wt % to about 4.5 wt/wt %, from about 0.5 wt/wt % to about 5 wt/wt %, from about 0.5 wt/wt % to about 6 wt/wt %, from about 0.5 wt/wt % to about 7 wt/wt %, from about 0.5 wt/wt % to about 8 wt/wt %, from about 0.5 wt/wt % to about 9 wt/wt %, from about 0.5 wt/wt % to about 10 wt/wt %, from about 1 wt/wt % to about 2 wt/wt %, from about 1 wt/wt % to about 2.5 wt/wt %, from about 1 wt/wt % to about 3 wt/wt %, from about 1 wt/wt % to about 3.5 wt/wt %, from about 1 wt/wt % to about 4 wt/wt %, from about 1 wt/wt % to about 4.5 wt/wt %, from about 1 wt/wt % to about 5 wt/wt %, from about 1 wt/wt % to about 6 wt/wt %, from about 1 wt/wt % to about 7 wt/wt %, from about 1 wt/wt % to about 8 wt/wt %, from about 1 wt/wt % to about 9 wt/wt %, from about 1 wt/wt % to about 10 wt/wt %, from about 1.5 wt/wt % to about 2 wt/wt %, from about 1.5 wt/wt % to about 2.5 wt/wt %, from about 1.5 wt/wt % to about 3 wt/wt %, from about 1.5 wt/wt % to about 3.5 wt/wt %, from about 1.5 wt/wt % to about 4 wt/wt %, from about 1.5 wt/wt % to about 4.5 wt/wt %, from about 1.5 wt/wt % to about 5 wt/wt %, from about 1.5 wt/wt % to about 6 wt/wt %, from about 1.5 wt/wt % to about 7 wt/wt %, from about 1.5 wt/wt % to about 8 wt/wt %, from about 1.5 wt/wt % to about 9 wt/wt %, from about 1.5 wt/wt % to about 10 wt/wt %, from about 2 wt/wt % to about 2.5 wt/wt %, from about 2 wt/wt % to about 3 wt/wt %, from about 2 wt/wt % to about 3.5 wt/wt %, from about 2 wt/wt % to about 4 wt/wt %, from about 2 wt/wt % to about 4.5 wt/wt %, from about 2 wt/wt % to about 5 wt/wt %, from about 2 wt/wt % to about 6 wt/wt %, from about 2 wt/wt % to about 7 wt/wt %, from about 2 wt/wt % to about 8 wt/wt %, from about 2 wt/wt % to about 9 wt/wt %, from about 2 wt/wt % to about 10 wt/wt %, from about 2.5 wt/wt % to about 3 wt/wt %, from about 2.5 wt/wt % to about 3.5 wt/wt %, from about 2.5 wt/wt % to about 4 wt/wt %, from about 2.5 wt/wt % to about 4.5 wt/wt %, from about 2.5 wt/wt % to about 5 wt/wt %, from about 2.5 wt/wt % to about 6 wt/wt %, from about 2.5 wt/wt % to about 7 wt/wt %, from about 2.5 wt/wt % to about 8 wt/wt %, from about 2.5 wt/wt % to about 9 wt/wt %, from about 2.5 wt/wt % to about 10 wt/wt %, from about 3 wt/wt % to about 4 wt/wt %, from about 3 wt/wt % to about 4.5 wt/wt %, from about 3 wt/wt % to about 5 wt/wt %, from about 3 wt/wt % to about 6 wt/wt %, from about 3 wt/wt % to about 7 wt/wt %, from about 3 wt/wt % to about 8 wt/wt %, from about 3 wt/wt % to about 9 wt/wt %, from about 3 wt/wt % to about 10 wt/wt %, from about 3.5 wt/wt % to about 4 wt/wt %, from about 3.5 wt/wt % to about 4.5 wt/wt %, from about 3.5 wt/wt % to about 5 wt/wt %, from about 3.5 wt/wt % to about 6 wt/wt %, from about 3.5 wt/wt % to about 7 wt/wt %, from about 3.5 wt/wt % to about 8 wt/wt %, from about 3.5 wt/wt % to about 9 wt/wt %, from about 3.5 wt/wt % to about 10 wt/wt %, from about 4 wt/wt % to about 4.5 wt/wt %, from about 4 wt/wt % to about 5 wt/wt %, from about 4 wt/wt % to about 6 wt/wt %, from about 4 wt/wt % to about 7 wt/wt %, from about 4 wt/wt % to about 8 wt/wt %, from about 4 wt/wt % to about 9 wt/wt %, from about 4 wt/wt % to about 10 wt/wt %, from about 4.5 wt/wt % to about 5 wt/wt %, from about 4.5 wt/wt % to about 6 wt/wt %, from about 4.5 wt/wt % to about 7 wt/wt %, from about 4.5 wt/wt % to about 8 wt/wt %, from about 4.5 wt/wt % to about 9 wt/wt %, from about 4.5 wt/wt % to about 10 wt/wt %, from about 5 wt/wt % to about 6 wt/wt %, from about 5 wt/wt % to about 7 wt/wt %, from about 5 wt/wt % to about 8 wt/wt %, from about 5 wt/wt % to about 9 wt/wt %, from about 5 wt/wt % to about 10 wt/wt %, from about 6 wt/wt % to about 7 wt/wt %, from about 6 wt/wt % to about 8 wt/wt %, from about 6 wt/wt % to about 9 wt/wt %, from about 6 wt/wt % to about 10 wt/wt %, from about 7 wt/wt % to about 8 wt/wt %, from about 7 wt/wt % to about 9 wt/wt %, from about 8 wt/wt % to about 10 wt/wt %, from about 8 wt/wt % to about 9 wt/wt %, from about 8 wt/wt % to about 10 wt/wt %, or from about 9 wt/wt % to about 10 wt/wt % of a Compound of the Disclosure.

In some embodiments, a Composition of the Disclosure comprises about 50 wt/wt % of a Compound of the Disclosure. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure. In some embodiments, the composition comprises about 0.1 wt/wt %, about 0.5 wt/wt %, about 1 wt/wt %, about 1.5 wt/wt %, about 2 wt/wt %, about 2.5 wt/wt %, about 3 wt/wt %, about 3.5 wt/wt %, about 4 wt/wt %, about 4.5 wt/wt %, about 5 wt/wt %, about 5.5 wt/wt %, about 6 wt/wt %, about 6.5 wt/wt %, about 7 wt/wt %, about 7.5 wt/wt %, about 8 wt/wt %, about 8.5 wt/wt %, about 9 wt/wt %, or about 10 wt/wt % of a Compound of the Disclosure.

In another embodiment, a Composition of the Disclosure comprises a Compound of the Disclosure and one or more elastomers.

The term “elastomer” as used herein is a polymer with viscoelasticity (i.e., having both viscosity and elasticity) that typically has low intermolecular forces, low Young's modulus, and high failure strain. Elastomers can typically be cross-linked by heating in the presence of one or more cross-linking agents, a process called curing or vulcanization. Rubber is one type of elastomer. Non-limiting types of rubber include natural rubber (NR), synthetic rubber, and blends thereof. The term “natural rubber” as used herein refers to a naturally occurring elastomer that can be obtained from Hevea rubber trees. Non-limiting types of synthetic rubbers include unsaturated rubbers, saturated rubbers, rubbers with fluoro and fluoroalkyl or fluoroalkoxy substituent groups on the polymer chain (FKM), silicone rubbers (Q), and blends thereof. Non-limiting examples of unsaturated rubbers include polyisoprene rubber (IR), butyl rubber (IIR), polybutadiene rubber (BR), styrene-isoprene-butadiene rubber (SIBR), styrene butadiene rubber (SBR), nitrile butadiene rubber (NBR), chloroprene rubber (CR), ethylene propylene diene rubber (EPDM), and blends thereof. These unsaturated rubbers undergo cyclization and crosslinking reactions that lead to hardening of the aged part. As oxidation occurs, these vulcanizates harden and eventually become brittle products. Partial oxidation of vulcanizates leads to losses in performance when used in applications such as vehicle tire sidewalls. Saturated rubbers are rubbers that do not contain C═C unsaturation and include, but are not limited to, acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), rubbers with fluoro and fluoroalkyl or fluoroalkoxy substituent groups on the polymer chain (FKM), silicone rubber (Q), and blends thereof.

In some embodiments, the natural rubber comprises rubber derived from an alternative rubber plant. The term “natural rubber comprises rubber derived from an alternative rubber plant” as used herein refers to a naturally occurring elastomer that can be obtained from “non-Hevea” sources. In some embodiments, the alternative rubber plant is Parthenium argentatum (guayule) or Taraxacum kok-saghyz (Russian dandelion).

In some embodiments, the one or more elastomers further comprises recycled rubber. The term “recycled rubber” as used herein refers to an elastomer that has been reclaimed from scrap materials such as used tires.

In some embodiments, a Composition of the Disclosure comprises from about 15 wt/wt % to about 85 wt/wt % of one or more elastomers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more elastomers.

In some embodiments, a Composition of the Disclosure comprises about 50 wt/wt % of one or more elastomers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more elastomers.

The term “phr” as used herein refers to parts per hundred parts of rubber by weight. The parts by weight of individual components are based on 100 parts by weight of the total mass of the one or more elastomers present in the composition.

In some embodiments, a Composition of the Disclosure comprises from about 1 phr to about 5 phr of a Compound of the Disclosure. In some embodiments, the composition comprises from about 0.01 phr to about 0.1 phr, from about 0.01 phr to about 0.5 phr, from about 0.01 phr to about 1 phr, from about 0.01 phr to about 2 phr, from about 0.01 phr to about 3 phr, from about 0.01 phr to about 4 phr, from about 0.01 phr to about 5 phr, from about 0.01 phr to about 7.5 phr, from about 0.01 phr to about 10 phr, from about 0.01 phr to about 20 phr, from about 0.1 phr to about 0.5 phr, from about 0.1 phr to about 1 phr, from about 0.1 phr to about 2 phr, from about 0.1 phr to about 3 phr, from about 0.1 phr to about 4 phr, from about 0.1 phr to about 5 phr, from about 0.1 phr to about 7.5 phr, from about 0.1 phr to about 10 phr, from about 0.1 phr to about 20 phr, from about 1 phr to about 2 phr, from about 1 phr to about 3 phr, from about 1 phr to about 4 phr, from about 1 phr to about 7.5 phr, from about 1 phr to about 10 phr, from about 1 phr to about 20 phr, from about 2 phr to about 3 phr, from about 2 phr to about 4 phr, from about 2 phr to about 5 phr, from about 2 phr to about 7.5 phr, from about 2 phr to about 10 phr, from about 2 phr to about 20 phr, from about 3 phr to about 4 phr, from about 3 phr to about 5 phr, from about 3 phr to about 7.5 phr, from about 3 phr to about 10 phr, from about 3 phr to about 20 phr, from about 4 phr to about 5 phr, from about 4 phr to about 7.5 phr, from about 4 phr to about 10 phr, from about 4 phr to about 20 phr, from about 5 phr to about 7.5 phr, from about 5 phr to about 10 phr, from about 5 phr to about 20 phr, from about 7.5 phr to about 10 phr, from about 7.5 phr to about 20 phr, or from about 10 phr to about 20 phr of a Compound of the Disclosure.

In some embodiments, a Composition of the Disclosure comprises about 3 phr of a Compound of the Disclosure. In some embodiments, the composition comprises about 0.01 phr, about 0.1 phr, about 0.5 phr, about 1 phr, about 2 phr, about 3 phr, about 4 phr, about 5 phr, about 7.5 phr, about 10 phr, or about 20 phr of a Compound of the Disclosure.

In some embodiments, a Composition of the Disclosure comprises a Compound of the Disclosure and one or more fillers.

The term “filler” as used herein is a substance that reinforces an elastomeric composition or gives an elastomeric composition other properties, including but not limited to expanding the volume of the composition. Non-limiting examples of fillers include carbon black, silica, kaolin, calcium silicate, talc, carbon nanotubes (CNT), carbon fibers (HCF), graphite, graphenes, aluminosilicates, starch, and fibers, and combinations thereof.

In some embodiments, the filler is derived from natural sources. For example, silica may be derived from rice husks.

In some embodiments, a Composition of the Disclosure comprises from about 15 wt/wt % to about 85 wt/wt % of one or more fillers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more fillers.

In some embodiments, a Composition of the Disclosure comprises about 50 wt/wt % of one or more fillers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more fillers.

In some embodiments, a Composition of the Disclosure comprises from about 30 phr to about 500 phr of one or more fillers. In some embodiments, the composition comprises from about 30 phr to about 50 phr, from about 30 phr to about 100 phr, from about 30 phr to about 150 phr, from about 30 phr to about 200 phr, from about 30 phr to about 250 phr, from about 30 phr to about 300 phr, from about 30 phr to about 350 phr, from about 30 phr to about 400 phr, from about 30 phr to about 450 phr, from about 30 phr to about 500 phr, from about 50 phr to about 100 phr, from about 50 phr to about 150 phr, from about 50 phr to about 200 phr, from about 50 phr to about 250 phr, from about 50 phr to about 300 phr, from about 50 phr to about 350 phr, from about 50 phr to about 400 phr, from about 50 phr to about 450 phr, from about 50 phr to about 500 phr, from about 100 phr to about 150 phr, from about 100 phr to about 200 phr, from about 100 phr to about 250 phr, from about 100 phr to about 300 phr, from about 100 phr to about 350 phr, from about 100 phr to about 400 phr, from about 100 phr to about 450 phr, from about 100 phr to about 500 phr, from about 150 phr to about 200 phr, from about 150 phr to about 250 phr, from about 150 phr to about 300 phr, from about 150 phr to about 350 phr, from about 150 phr to about 400 phr, from about 150 phr to about 450 phr, from about 150 phr to about 500 phr, from about 200 phr to about 250 phr, from about 200 phr to about 300 phr, from about 200 phr to about 350 phr, from about 200 phr to about 400 phr, from about 200 phr to about 450 phr, from about 200 phr to about 500 phr, from about 250 phr to about 300 phr, from about 250 phr to about 350 phr, from about 250 phr to about 400 phr, from about 250 phr to about 450 phr, from about 250 phr to about 500 phr, from about 300 phr to about 350 phr, from about 300 phr to about 400 phr, from about 300 phr to about 450 phr, from about 300 phr to about 500 phr, from about 350 phr to about 400 phr, from about 350 phr to about 450 phr, from about 350 phr to about 500 phr, from about 400 phr to about 450 phr, from about 400 phr to about 500 phr, or from about 450 phr to about 500 phr of one or more fillers.

In some embodiments, a Composition of the Disclosure comprises about 300 phr of one or more fillers. In some embodiments, the composition comprises about 30 phr, about 50 phr, about 100 phr, about 150 phr, about 200 phr, about 250 phr, about 350 phr, about 400 phr, about 450 phr, or about 500 phr of one or more fillers.

In some embodiments, a Composition of the Disclosure comprises a Compound of the Disclosure and one or more rubber chemicals. The term “rubber chemicals” as used herein refers to a compound or substance used to facilitate the vulcanization of rubber. Rubber chemicals include, but are not limited to, vulcanizing agents, accelerators, activators, and pre-vulcanization inhibitors.

The term “vulcanization” as used herein refers to a process wherein cross-links are formed between elastomers to effect changes in the material properties of elastomers. In particular, vulcanization typically increases the rigidity and durability of elastomers. Vulcanization is carried out at room temperature or at elevated temperatures, depending on the nature of the elastomer(s), filler(s), and rubber chemical(s) being used. The term “curing” is also used in the art to describe this process.

The term “vulcanizing agent” as used herein refers to any substance that enables cross-linking between elastomers. Vulcanizing agents can enable cross-linking between separate polymer chains of an elastomer by various mechanisms, including, but not limited to, by formation of covalent bonds between the vulcanizing agent and two or more separate polymer chains or by generating radical species on separate polymer chains that can combine to form covalent bonds between the two polymer chains. Non-limiting examples of vulcanizing agents include sulfur, peroxides, vulcanized vegetable oil, factices and resins. Non-limiting examples of sulfur include octasulfur (S₈), cyclododecasulfur (S₁₂), and polymeric sulfur. Non-limiting examples of peroxides include benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), and 1,1-di(tert-butylperoxy)-3,3,5-trimethylcyclohexane (TMC). Nonlimiting examples of resins include bonding resins. The term “bonding resin” as used herein refers to a chemical such as resorcinol formaldehyde resins and phenolic resins that reacts with methylene donors (such as hexamethylenetetramine (HMTA) or hexamethoxymethyl melamine (HMMM)) to promote adhesion.

The term “accelerator” as used herein refers to any substance that increases the kinetics of vulcanization. In some embodiments, accelerators enable vulcanization to be performed at lower temperatures and/or to use the vulcanization agent, e.g., sulfur, more efficiently. Non-limiting examples of accelerators include guanidines, thiazoles, sulfenamides, sulfenimides, thiurams, dithiocarbamates, xanthates, and thiophosphates. Non-limiting examples of guanidines include diphenylguanidine (DPG). Non-limiting examples of thiazoles include 2-mercaptobenzothiazole (MBT), zinc 2-mercaptobenzothiazole (ZMBT), mercaptobenzothiazole disulfide (MBTS), and N-tert-butyl-2-benzothiazole sulfenamide (TBSI). Non-limiting examples of sulfenamides include N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), and thiocarbamyl sulfenamide. Non-limiting examples of thiurams include dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), and tetramethylthiuram monosulfide (TMTM). Non-limiting examples of dithiocarbamates include zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), and zinc dibenzyldithiocarbamate (ZEBC).

The term “activator” as used herein refers to any substance that activates a vulcanizing agent and enables it to cross-link elastomers as described above. Activators may act via various mechanisms, including, but not limited to, by forming chemical complexes with accelerators or by coordinating to sulfur (when sulfur is used as a vulcanizing agent). Non-limiting examples of activators include metal oxides, acids, and metal complexes. Non-limiting examples of metal oxides include zinc oxide, magnesium oxide, and lead oxide. Non-limiting examples of acids include stearic acid and lauric acid. Non limiting examples of metal complexes include zinc ethylhexanoate.

The term “pre-vulcanization inhibitor” as used herein refers to compounds that delay the onset and/or the rate of vulcanization. These compounds are also referred to as “retarders.” Non-limiting examples of pre-vulcanization inhibitors include N-(cyclohexylthio)phthalimide (CTP), benzoic anhydride, salicylic anhydride, and phthalic anhydride.

In some embodiments, a Composition of the Disclosure comprises from about 15 wt/wt % to about 85 wt/wt % of one or more rubber chemicals. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more rubber chemicals.

In some embodiments, a Composition of the Disclosure comprises about 15 wt/wt % of one or more rubber chemicals. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more rubber chemicals.

In some embodiments, a Composition of the Disclosure comprises from about 1 phr to about 20 phr of one or more rubber chemicals. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more rubber chemicals.

In some embodiments, a Composition of the Disclosure comprises about 10 phr of one or more rubber chemicals. In some embodiments, the composition comprises about 0.1 phr, about 1 phr, about 5 phr, about 15 phr, about 20 phr, about 25 phr, about 30 phr, about 35 phr, or about 40 phr of one or more rubber chemicals.

The term “plasticizer” as used herein refers to a processing aid used to reduce the viscosity, increase the plasticity, and/or extend the volume of a composition. Plasticizers facilitate the process of mixing and forming a composition comprising an elastomer before the composition is vulcanized. Non-limiting examples of plasticizers include mineral oils (paraffinic, aromatic, or naphthenic), organic esters, resins, waxes, ester plasticizers, and naturally derived oils, such as soybean oil, vegetable oil, or orange oil.

In some embodiments, a Composition of the Disclosure comprises from about 15 wt/wt % to about 85 wt/wt % of one or more plasticizers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more plasticizers.

In some embodiments, a Composition of the Disclosure comprises about 15 wt/wt % of one or more plasticizers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more plasticizers.

In some embodiments, a Composition of the Disclosure comprises from about 1 phr to about 20 phr of one or more plasticizers. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more plasticizers.

In some embodiments, a Composition of the Disclosure comprises about 10 phr of one or more plasticizers. In some embodiments, the composition comprises about 0.1 phr, about 1 phr, about 5 phr, about 15 phr, about 20 phr, about 25 phr, about 30 phr, about 35 phr, or about 40 phr of one or more plasticizers.

In some embodiments, a Composition of the Disclosure further comprises a second antidegradant that is not a Compound of the Disclosure. In some embodiments, the second antidegradant is an antioxidant. In some embodiments, the second antidegradant is an antiozonant. Non-limiting examples of antidegradants include paraphenylenediamines (PPDs), trimethyl-dihydroquinolines (TMQs), phenolics, alkylated diphenylamines (DPAs), diphenylamine-ketone condensates, and natural antidegradants. Non-limiting examples of PPDs include N¹-(4-methylpentan-2-yl)-N⁴-phenylbenzene-1,4-diamine (6PPD), N-(1,4-dimethylpentyl)-N′-phenyl-p-phenylenediamine (7PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), and N—N′-dioctyl-p-phenylenediamine (88PD). Non-limiting examples of TMQs include 2,2,4-trimethyl-1,2-dihydroquinoline and oligomers or polymers thereof.

In some embodiments, a Composition of the Disclosure comprises from about 15 wt/wt % to about 85 wt/wt % of a second antidegradant. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of a second antidegradant.

In some embodiments, a Composition of the Disclosure comprises about 15 wt/wt % of a second antidegradant. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a second antidegradant.

In some embodiments, a Composition of the Disclosure comprises from about 1 to about 5 phr of a second antidegradant. In some embodiments, the composition comprises from about 0.001 phr to about 0.01 phr, from about 0.001 phr to about 0.1 phr, from about 0.001 phr to about 1 phr, from about 0.001 phr to about 5 phr, from about 0.001 phr to about 7.5 phr, from about 0.001 phr to about 10 phr, from about 0.01 phr to about 0.1 phr, from about 0.01 phr to about 1 phr, from about 0.01 phr to about 5 phr, from about 0.01 phr to about 7.5 phr, from about 0.01 phr to about 10 phr, from about 0.1 phr to about 1 phr, from about 0.1 phr to about 5 phr, from about 0.1 phr to about 7.5 phr, from about 0.1 phr to about 10 phr, from about 1 phr to about 7.5 phr, from about 1 phr to about 10 phr, from about 5 phr to about 7.5 phr, from about 5 phr to about 10 phr, or from about 7.5 phr to about 10 phr.

In some embodiments, a Composition of the Disclosure comprises about 3 phr of a second antidegradant. In some embodiments, the composition comprises about 0.001 phr, about 0.01 phr, about 0.1 phr, about 1 phr, about 2 phr, about 4 phr, about 5 phr, about 7.5 phr, or about 10 phr of a second antidegradant.

In some embodiments, the disclosure provides a composition comprising a Compound of the Disclosure and one or more carriers. The term “carrier” as used herein refers to a solid that can adsorb a liquid while retaining the general properties of a solid at room temperature. In some embodiments, the carrier is an inert material. In some embodiments, the carrier has a high surface area. In some embodiments, the carrier comprises particles with diameters of less than 500 microns.

In some embodiments, the composition comprises from about 15 wt/wt % to about 85 wt/wt % of one or more carriers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more carriers.

In some embodiments, the composition comprises about 15 wt/wt % of one or more carriers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more carriers.

The present disclosure also provides processes for preparing a composition comprising a Compound of the Disclosure and one or more carriers, the process comprising admixing the compound and the one or more carriers.

The present disclosure also provides lubricant compositions comprising a Compound of the Disclosure and a lubricant. Non-limiting examples of lubricants include mineral oil, higher molecular weight petroleum distillates such as aromatic, naphthenic, and paraffinic distillates, synthetic oils such as polyalpha-olefin (PAO), synthetic esters, polyalkylene glycols (PAG), phosphate esters, perfluoropolyether (PFPE), alkylated naphthalenes (AN), silicate esters, ionic fluids, and multiply alkylated cyclopentanes (MAC), solid lubricants such as polytetrafluoroethylene (PTFE), graphite, hexagonal boron nitride, molybdenum disulfide, tungsten disulfide, aqueous lubricants such as hydrated brush polymers, and biolubricants such as triglyceride esters, high oleic canola oil, castor oil, palm oil, sunflower seed oil, and rapeseed oil.

The present disclosure also provides combustible fuel compositions comprising a combustible fuel and a Compound of the Disclosure. Non-limiting examples of combustible fuel include gasoline, diesel, kerosene, liquefied petroleum gas, synthetic fuel, and biodiesel.

The present disclosure also provides fuel additive compositions comprising a fuel additive and a Compound of the Disclosure. Non-limiting examples of fuel additives include oxygenates such as alcohols and ethers, antioxidants, stabilizers, detergents, antiknock agents, lead scavengers, fuel dyes, viscosity modifiers, and butyl rubber. In some embodiments, the butyl rubber is in the form of polyisobutylene succinimide. In some embodiments, the butyl rubber is added as a detergent to prevent fouling of diesel fuel injectors.

### Vulcanized Elastomeric Articles

The present disclosure also provides vulcanized elastomeric articles comprising a Compound of the Disclosure. The term “vulcanized elastomeric article” refers to an article that is made by forming a composition comprising an elastomer into a specific shape and vulcanizing the composition to provide the article.

The present disclosure also provides vulcanized elastomeric articles comprising a composition described herein.

The present disclosure also provides vulcanized elastomeric articles prepared using a Compound of the Disclosure.

The present disclosure also provides vulcanized elastomeric articles prepared using a composition described herein.

In some embodiments, the vulcanized elastomeric article is a tire. In some embodiments, the tire is a passenger vehicle tire, a light truck tire, a heavy truck or bus tire, a motorcycle tire, an agriculture tire, an earthmover tire, an airplane tire, or a racing tire.

In some embodiments, the vulcanized elastomeric article is a component of a tire. In some embodiments, the component is a bead, a belt, a body ply, an inner liner, a sidewall, an undertread, or a tread.

In some embodiments, the vulcanized elastomeric article is a rubber overshoe, a sealing strip, an acoustic panel, an air spring, a bellow, a membrane, a tactile sensor, a crash pad, a hose, a conveyor belt, or a flooring.

### Processes

The present disclosure also provides processes for preparing a vulcanized elastomeric article, the process comprising:
(a) forming a composition described herein into a formed shape; and
(b) vulcanizing the formed shape,
   to provide a vulcanized elastomeric article.

In some embodiments, the vulcanizing is performed at an average temperature of from about 140° C. to about 160° C. In some embodiments, the vulcanizing is performed at an average temperature of from about 80° C. to about 100° C., from about 80° C. to about 120° C., from about 80° C. to about 140° C., from about 80° C. to about 160° C., from about 80° C. to about 180° C., from about 80° C. to about 200° C., from about 100° C. to about 120° C., from about 100° C. to about 140° C., from about 100° C. to about 160° C., from about 100° C. to about 180° C., from about 100° C. to about 200° C., from about 120° C. to about 140° C., from about 120° C. to about 160° C., from about 120° C. to about 180° C., from about 120° C. to about 200° C., from about 140° C. to about 180° C., from about 140° C. to about 200° C., from about 160° C. to about 180° C., from about 160° C. to about 200° C., or from about 180° C. to about 200° C.

In some embodiments, the vulcanizing is performed at an average temperature of about 150° C. In some embodiments, the vulcanizing is performed at an average temperature of about 80° C., about 100° C., about 120° C., about 140° C., about 160° C., about 180° C., or about 200° C.

The present disclosure also provides processes for retreading tires, the process comprising:
(a) applying a composition described herein to a tire;
(b) disposing a pre-vulcanized tread around the tire;
(c) disposing a curing envelope around the tire; and
(d) vulcanizing the tire.

### Kits

The present disclosure also provides kits comprising a composition described herein, packaged in a manner, e.g., in a container, that facilitates use of the composition to practice the processes and/or methods of the present disclosure. In some embodiments, the kit comprises a composition described herein and instructions for using the composition in a vulcanizable elastomeric composition. In some embodiments, the kit comprises a composition described herein and instructions for using the composition to prepare a vulcanized elastomeric article. The composition may be packaged in any suitable container, such as a sealed bottle or vessel, with a label affixed to the container or included in the kit that describes the composition and proper use thereof.

### EXAMPLES

### Example 1

### Synthesis of N-(sec-butyl)-1H-indol-5-amine (Compound 1)

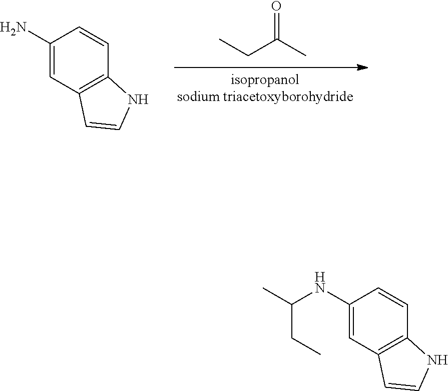

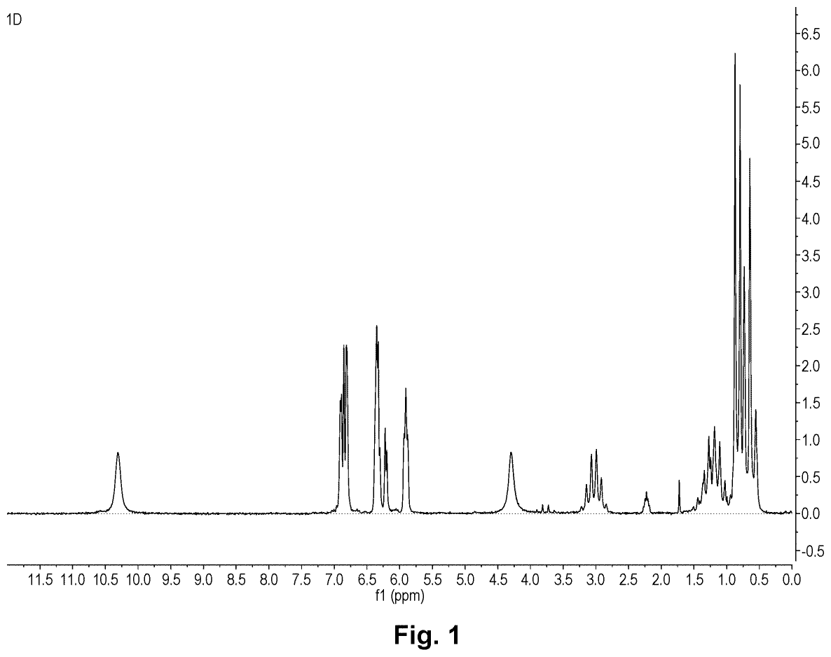

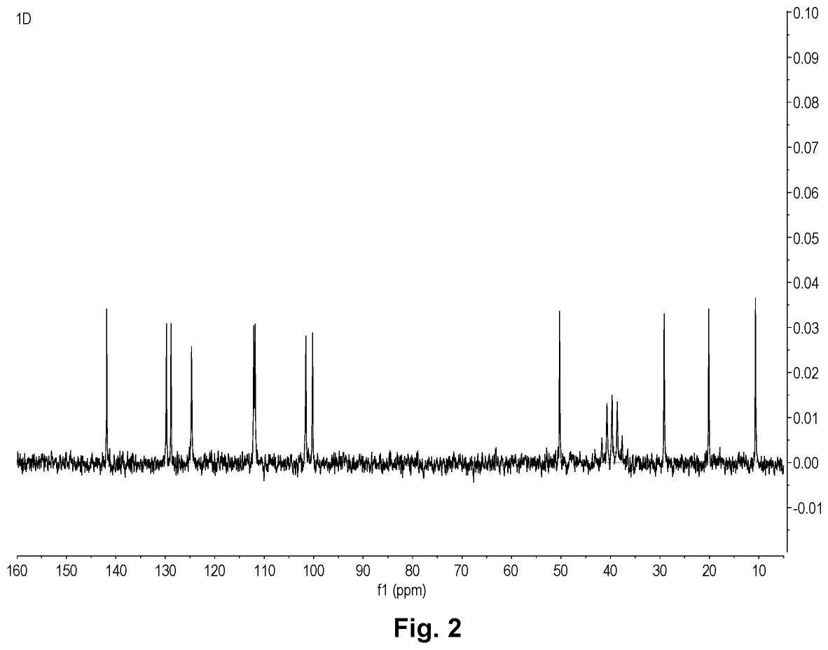

5-aminoindole (20.08 g; 151.9 mmol), isopropanol, (117 mL), and methyl ethyl ketone (47.7 mL; 532 mmol) were added to a 500-mL round bottom flask fitted with a stir-bar, an alcohol thermometer, and a reflux condenser. The reaction mixture was stirred and heated with an oil bath (oil temperature was set to 90° C.) under N₂ protection. Once the reflux temperature was attained (internal temperature=approx. 80° C.), the temperature was held for 7.5 hours to form the imine intermediate. Upon cooling to room temperature overnight, the flask was immersed in a cold tap water bath. Sodium triacetoxyborohydride (42.5 g; 201 mmol), was added in portions over 10 min (apex temperature=31° C.). Once the addition of sodium triacetoxyborohydride was complete, isopropanol (5 mL) was used to rinse the sides of the funnel. The reaction mixture was stirred for 7 min, then the cooling bath was removed. The reaction mixture was stirred without additional cooling or heating for about 2.5 hours. The flask was cooled with a cold tap water bath. Aqueous 2 M NaOH (102 mL) was added by portions over 8-9 min under vigorous stirring. Ethyl acetate (70 mL) was added (no exotherm was observed). The 2-phase system was transferred to a separatory funnel. Additional ethyl acetate (180 mL) was used to rinse the flask and added to the separatory funnel, along with another portion of aqueous 2 M NaOH (102 mL). The separatory funnel was shaken and allowed to settle. The aqueous phase was discarded. The organic phase was washed with aqueous 2 M NaOH (102 mL), deionized water (102 mL), and aqueous saturated NaCl (102 mL). The organic solution was dried over anhydrous MgSO₄, filtered, and stripped of volatiles under reduced pressure (rotary evaporator; water bath=55° C.). The obtained dark liquid was dried on the rotary evaporator (60° C.; <2 mbar) for 1.5 hours to provide 27.2 g (95% of the theoretical with respect to 5-aminoindole) of N-(sec-butyl)-1H-indol-5-amine (Compound 1) as a dark oil. Compound 1 was characterized by ¹H and ¹³C nuclear magnetic resonance (NMR) spectroscopy, as shown in FIGS. 1 and 2, respectively, and was determined by LC-MS/UV to have a purity of >93.5%.

### Example 2

### Synthesis of N-(sec-butyl)indolin-5-amine (Compound 2)

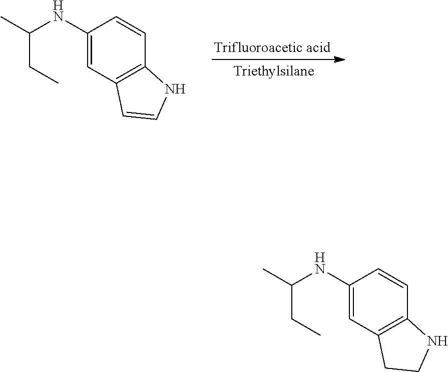

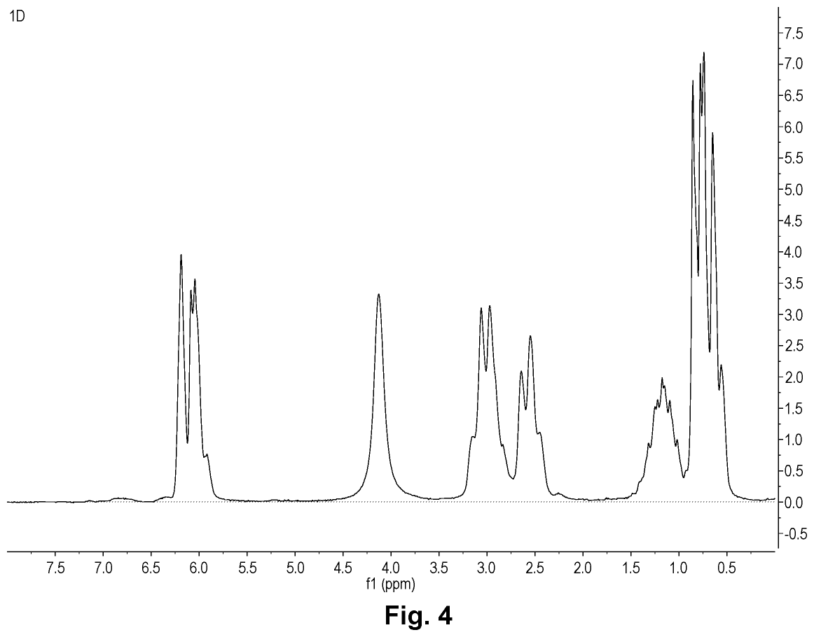

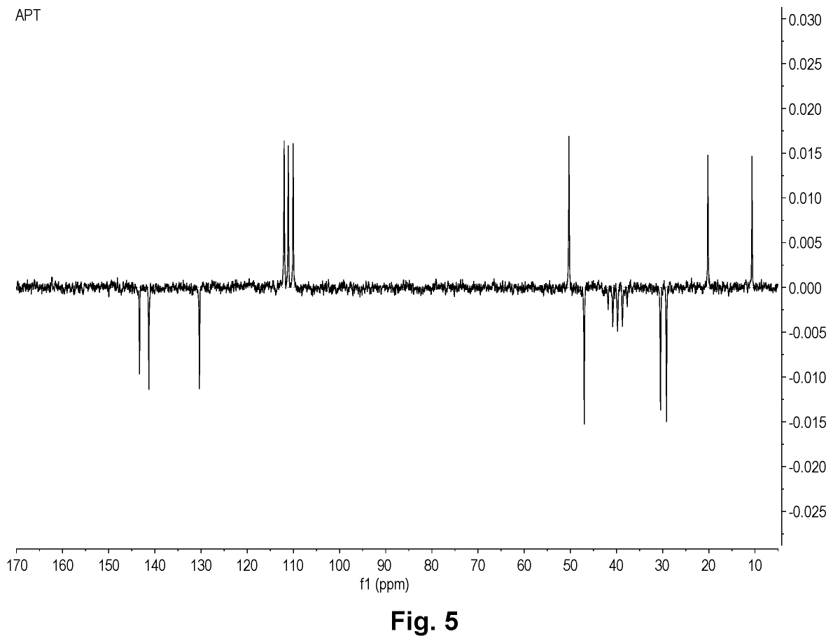

Compound 1 (1.03 g; 5.47 mmol) was added to a 100-mL round bottom flask fitted with a stir-bar. The flask was protected with N₂ blanket and stirred. Trifluoroacetic acid (5.7 mL) was added. After about 18-24 hours, the flask was cooled with a cold tap water bath. Triethylsilane (1.8 mL; 11.3 mmol) was added via a syringe. No exotherm was observed. The obtained two-phase system was stirred at room temperature for about 22 hours. Silica gel TLC (50% ethyl acetate in hexanes) indicated complete disappearance of the starting material at R_{f}=0.7, and formation of intense dot at R_{f}=0.2. The flask was chilled with cold tap water bath. Aqueous 2 mol/L NaOH (43 mL) was slowly added to the flask. Ethyl acetate (26 mL) was added and the 2-phase system was stirred vigorously until complete dissolution of the oily material. The aqueous layer was discarded. The organic layer was washed with deionized water (20 mL) and brine (20 mL), then stripped of its volatiles under reduced pressure (rotary evaporator; water bath=50° C.). The crude material (black liquid; approx. 1.37 g) was diluted with 50% ethyl acetate in hexanes (3.0 mL). The obtained solution was loaded into a 1-inch inner diameter chromatography column previously prepared with silica gel 60 (approx. 7.5 inches height) and 50% ethyl acetate in hexanes. The column was eluted with 50% ethyl acetate in hexanes, then with 100% ethyl acetate. Each fraction was analyzed by silica gel TLC (50% ethyl acetate in hexanes). The fractions containing R_{f}=0.2 were collected, combined, and stripped of volatiles under reduced pressure (rotary evaporator; water bath=50° C.). Isolated yield=0.492 g as a dark-purple oil (47% based on Compound 1). Compound 2 was characterized by ¹H and ¹³C NMR, as shown in FIGS. 4 and 5, respectively, and was determined by LC-MS to have a purity of 96.68%.

Compound 2 can also be synthesized by reductive alkylation of 5-nitroindoline, as described below:

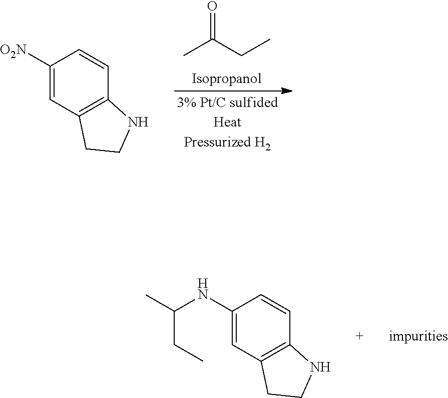

A 300-mL Paar autoclave was charged with 5-nitroindoline (5.0 grams; 30.46 mmol), isopropanol (150 mL), methyl ethyl ketone (4.5 mL; 50.2 mmol), and 3% platinum on carbon sulfided (0.1 g). The reaction mixture was heated to 120° C. under pressurized H₂ (250 PSI) for 3 hours. LC-UV/MS of the crude mixture indicated presence of several compounds including the desired product Compound 2.

### Example 3

### Synthesis of N-(1-phenylethyl)-1H-indol-5-amine (Compound 19)

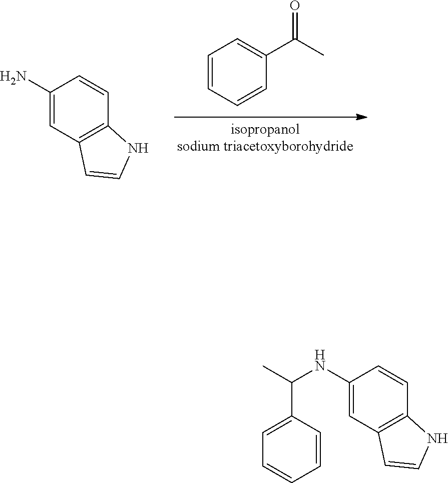

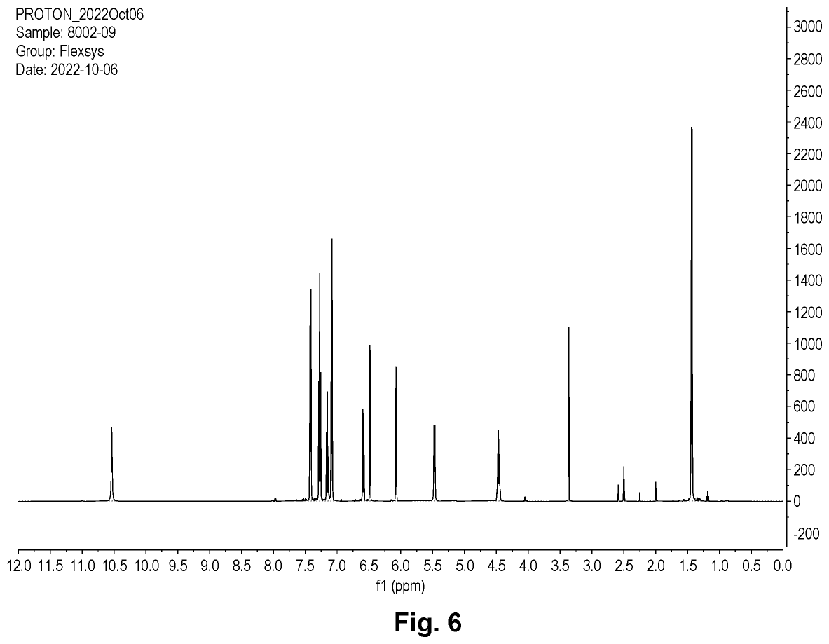

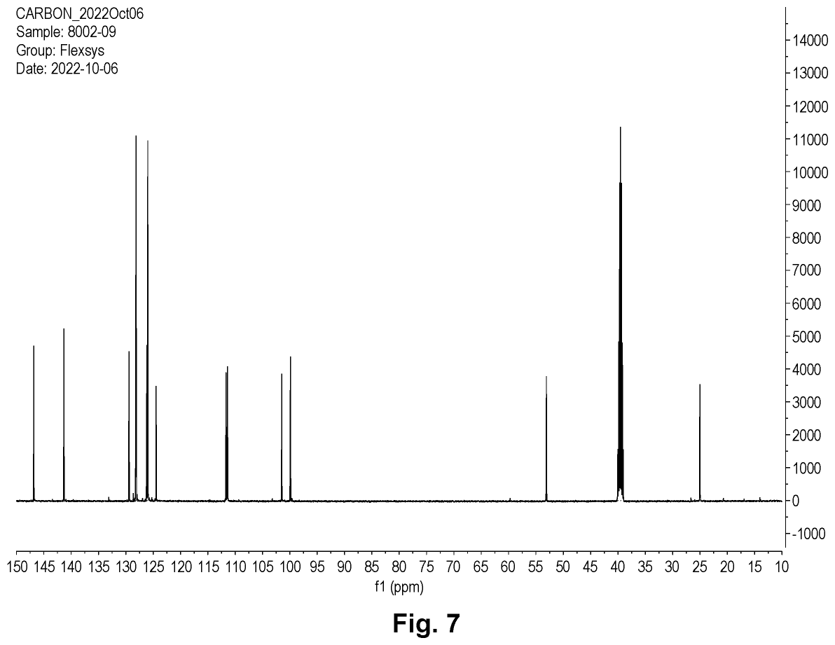

To a 100-mL round bottom flask fitted with a stir-bar and a reflux condenser was loaded 5-aminoindole (2.425 g; 18.35 mmol) and isopropanol (11 mL). The mixture was stirred under N₂ protection and acetophenone (2.8 mL; 24.0 mmol) was added. The reaction mixture was stirred at room temperature for 1 hour, refluxed for 3 hours (oil bath=91° C.), then allowed to cool to room temperature overnight. Silica gel TCL (20% ethyl acetate in hexanes) indicated formation of a spot at R_{f}=0.35. The spots at R_{f}=0.15 (5-aminoindole) and 0.60 (acetophenone) were still quite intense. The reaction mixture was refluxed for additional time (6-6.5 hours) then the flask was cooled with a cold tap water bath. Sodium triacetoxyborohydride (4.67 g; 22.0 mmol) was added by portions over 10-11 minutes. The cooling bath was removed. The reaction mixture was stirred at room temperature overnight (approx. 16 hours). Aqueous 1 mol/L NaOH (68 mL) was added portion-wise to the flask. The mixture was combined with ethyl acetate (50 mL) in a separatory funnel. After shaking and decanting, the aqueous phase was discarded. The organic phase was washed with aqueous 1 mol/L NaOH (25 mL), aqueous dilute NaCl (50 mL), aqueous saturated NaCl (25 mL), was dried over anhydrous MgSO4, filtered, and finally stripped of volatiles under reduced pressure (rotary evaporator; water bath=50° C.). The obtained dark oil (4.29 g) was dissolved in toluene (5.5 mL). The resulting solution was loaded into a 41 mm inner diameter chromatography column previously prepared with silica gel 60 and eluent 20% ethyl acetate in hexanes (22-23 cm height of silica gel). The column was eluted with the eluent. The portions (approx. 30 mL each) were analyzed by silica gel TLC (20% ethyl acetate in hexanes). The portions containing the desired product (R_{f}=0.35) were collected, combined, and stripped of volatiles under reduced pressure (rotary evaporator; water bath=55° C.). Further drying on the rotary evaporator (water bath=55° C.; 1-6 mbars; 15 min) afforded 2.33 g of a clear light-yellow oil, which darkened upon standing at ambient conditions (proton NMR showed the presence of residual AcOEt). The flask containing the material was immerged into a sand bath. Drying in the vacuum oven at 70° C. for several hours, then at room temperature overnight afforded Compound 19 as a light blue gooey material (2.1 g; 48% isolated yield with respect to 5-aminoindole). Compound 19 was characterized by ¹H and ¹³C NMR, as shown in FIGS. 6 and 7, respectively, and was determined by LC-MS to have a purity of 93.7%.

### Example 4

### Use of Compound 1 in Sidewall Formulations

A rubber compound (Example #1) was prepared using Compound 1 as an antidegradant in a sidewall formulation. As shown in Table 1, the rubber compound is a 50/50 blend of natural rubber and butadiene rubber with carbon black of the N500 series. The vulcanization properties of the compound were characterized by a moving die rheometer and summarized in Table 2.

TABLE 1

| Material | Example #1 |
|---|---|
| Natural rubber (SIR-10) | 50 |
| Butadiene rubber (CB24) | 50 |
| N-550 Carbon Black | 50 |
| Stearic acid | 2 |
| Calsol 8240 (Naphthenic Oil) | 6.25 |
| Zinc oxide | 4 |
| Sulfur | 1.5 |
| TBBS | 0.6 |
| Compound 1 | 1.41 |
| Total parts per hundred rubber (phr) | 165.76 |

TABLE 2

| | Example #1 |
|---|---|
| Temperature (° C.) | 150 |
| ML (dNm) | 2.58 |
| MH (dNm) | 15 |
| Ts2 (min) | 5.94 |
| T10 (min) | 5.6 |
| T50 (min) | 7.64 |
| T90 (min) | 12.09 |

### Example 5

### Antiozonant Performance of Compound 1 in Sidewall Formulations

The rubber compound prepared in Example 2 (Example #1) was cured at 150° C. to a state equivalent to its t90 time. The ozone testing specimens were died out from vulcanized tensile sheets by using a die followed the American Society for Testing and Materials (ASTM) D1329 standard. The ozone testing specimens were stored at ambient environment for more than 24 hours prior to antiozonant performance testing in an ozone chamber, which was maintained at an ozone concentration of 10±2 parts per hundred million (pphm) and 40±2° C. The ozone testing specimens were simultaneously relaxed and extended within ozone chamber under three different conditions: static, intermittent, and dynamic. The ozone testing specimens were maintained at 25% strain during static exposure and alternately relaxed and extended to 25% strain at a rate of 90 cycles per minute during dynamic exposure. The intermittent test alternated 18 minutes of dynamic exposure and 102 minutes of static exposure. Exposure was in increments of 16 hours followed by at least one hour of relaxation after removal from the ozone chamber and prior to measurement of force at a strain of 100%.

Antiozonant performance of antidegradant formulated compounds were evaluated by percentage of force retention. Percentage of force retention is defined by equation 1:

$$Percentage of force retention = \frac{F}{{F}_{0}} * 100 \% \qquad Equation 1$$

wherein F₀ is the force at a strain of 100% prior to ozone testing and F is the force at a strain of 100% after samples are aged in ozone chamber for 16-hour cycle at static, intermittent, or dynamic exposure. Higher percentage of force retention is an indication of better antiozonant performance of an antidegradant compound incorporated in the vulcanized rubber compound.

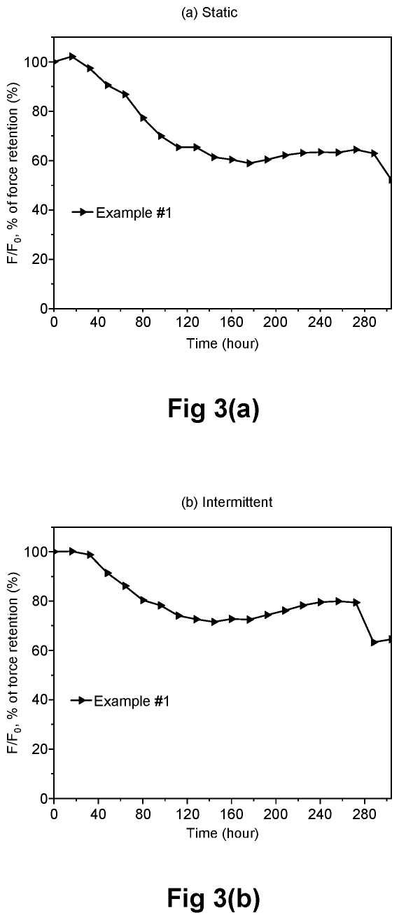

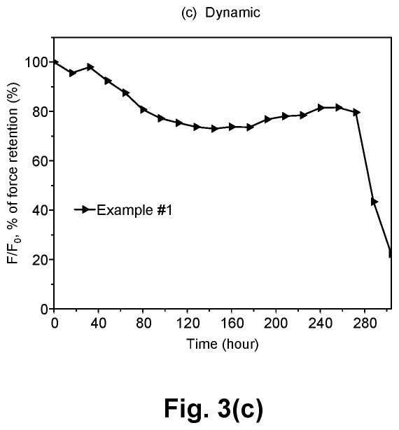

The percentage of force retention results at static, intermittent, and dynamic conditions were plotted as a function of ozone aging time in FIGS. 3(a)-3(c).

### Example 6

### Oxidative Induction Time (OIT) Performance of Compounds of the Disclosure

In order to demonstrate the antioxidant efficacy of Compounds of the Disclosure, the oxidative induction time (OIT) of representative compounds were evaluated. OIT is measured according to a procedure carried out in a differential scanning calorimeter (DSC) and is used to predict thermo-oxidative performance of a material. In this procedure, each sample is held in a sample cell and heated under a nitrogen atmosphere to a preselected temperature. Oxygen is then introduced to the sample cell and the length of time before the onset of degradation, as seen by the initiation of increase in heat flow, is measured.

0.5 wt % of a Compound of the Disclosure, or no antidegradant (blank) was mixed with polyisoprene and heated isothermally at 150° C. or 160° C. in 02. The peak time of OIT testing in minutes is shown in Table 3. As indicated by the data in Table 3, Compounds of the Disclosure demonstrate antioxidant performance compared against blank control and indicates utility in rubber and other applications that can benefit from an active antioxidant.

TABLE 3

Peak time of OIT testing (minutes)

| Material | 150° C. | 160° C. |
|---|---|---|
| Blank | 1.8 | 1.4 |
| Compound 1 | 41.4 | 19.2 |
| Compound 2 | 35.5 | 14.1 |
| Compound 19 | 15.7 | 6.9 |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

What is claimed is:

1. A composition comprising:
(i) a compound having Formula (I): 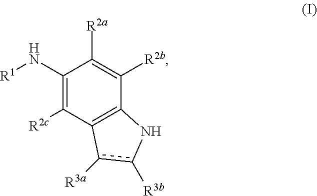
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{2c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- or 6-membered heterocyclyl;
R⁴ is selected from the group consisting of hydrogen and C₁-C₉ alkyl; and
R⁵ is selected from the group consisting of hydrogen and C₁-C₉ alkyl; and
(ii) one or more elastomers; or
(iii) one or more fillers; or
(iv) one or more rubber chemicals; or
(v) one or more plasticizers; or
(vi) a second antidegradant; or
(vii) a combination of one or more elastomers, one or more fillers, one or more rubber chemicals, one or more plasticizers, and/or a second antidegradant.

2. The composition of claim 1, wherein [symbol] is a double bond.

3. The composition of claim 1, wherein [symbol] is a single bond.

4. The composition of any one of claims 1-3, wherein R¹ is selected from the group consisting of C₁-C₁₂ alkyl and optionally substituted phenyl.

5. The composition of any one of claims 1-4, wherein R¹ is phenyl.

6. The composition of any one of claims 1-4, wherein R¹ is selected from the group consisting of methyl, ethyl, propyl, isopropyl, tert-butyl, sec-butyl, iso-butyl, pentyl, hexyl, heptyl, octyl, nonyl, decyl, undecyl, and dodecyl.

7. The composition of claim 6, wherein R¹ is sec-butyl.

8. The composition of claim 6, wherein R¹ is isopropyl.

9. The composition of any one of claims 1-3, wherein:
R¹ is —CHR^{1a}R^{1b};
R^{1a} is selected from the group consisting of: 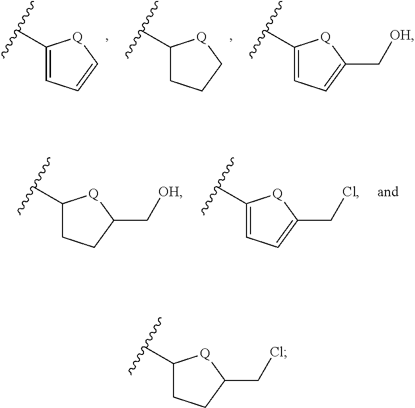
Q is selected from the group consisting of —NH—, —O—, and —S—; and
R^{1b} is selected from the group consisting of hydrogen and methyl.

10. The composition of any one of claims 1-9, wherein R^{2a}, R^{2b}, and R^{2c} are hydrogen.

11. The composition of any one of claims 1-10, wherein R^{3a} and R^{3b} are hydrogen.

12. The composition of claim 1, wherein the compound is selected from any one or more of the compounds of Table 1.

13. The composition of any one of claims 1-12, wherein the composition comprises one or more elastomers.

14. The composition of claim 13, wherein the composition comprises from about 15 wt/wt % to about 85 wt/wt % of the compound and from about 15 wt/wt % to about 85 wt/wt % of the one or more elastomers.

15. The composition of claim 13 or 14, wherein the one or more elastomers comprise natural rubber (NR).

16. The composition of claim 15, wherein the one or more elastomers comprises from about 5 wt/wt % to about 80 wt/wt % natural rubber (NR).

17. The composition of claim 15 or 16, wherein the natural rubber comprises rubber derived from an alternative rubber plant.

18. The composition of claim 17, wherein the alternative rubber plant is Parthenium argentatum (guayule) or Taraxacum kok-saghyz (Russian dandelion).

19. The composition of any one of claims 13-18, wherein the one or more elastomers comprise synthetic rubber.

20. The composition of claim 19, wherein the synthetic rubber comprises an unsaturated rubber, a saturated rubber, a rubber with fluoro and fluoroalkyl or fluoroalkoxy substituent groups on the polymer chain (FKM), a silicone rubber (Q), or a blend thereof.

21. The composition of claim 20, wherein the unsaturated rubber comprises polyisoprene rubber (IR), butyl rubber (IIR), polybutadiene rubber (BR), styrene butadiene rubber (SBR), nitrile butadiene rubber (NBR), chloroprene rubber (CR), ethylene propylene diene rubber (EPDM), or a blend thereof.

22. The composition of claim 21, wherein the unsaturated rubber comprises styrene butadiene rubber (SBR).

23. The composition of claim 22, wherein the one or more elastomers comprise from about 5 wt/wt % to about 80 wt/wt % styrene butadiene rubber (SBR).

24. The composition of any one of claims 21-23, wherein the unsaturated rubber comprises polybutadiene rubber (BR).

25. The composition of claim 24, wherein the one or more elastomers comprise from about 5 wt/wt % to about 80 wt/wt % polybutadiene rubber (BR).

26. The composition of any one of claims 21-25, wherein the unsaturated rubber comprises butyl rubber (IIR).

27. The composition of claim 26, wherein the one or more elastomers comprise from about 1 wt/wt % to about 30 wt/wt % butyl rubber (IIR).

28. The composition of any one of claims 20-27, wherein the saturated rubber comprises acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), or a blend thereof.

29. The composition of claims any one of claims 13-28, wherein the one or more elastomers further comprises recycled rubber.

30. The composition of any one of claims 1-29, wherein the composition comprises from about 0.1 phr to about 10 phr of the compound.

31. The composition of claim 30, wherein the composition comprises from about 0.5 phr to about 5 phr of the compound.

32. The compound of claim 31, wherein the composition comprises from about 1 phr to about 5 phr of the compound.

33. The composition of any one of claims 1-32, wherein the composition comprises one or more fillers.

34. The composition of claim 33, wherein the composition comprises from about 15 wt/wt % to about 85 wt/wt % of the compound and from about 15 wt/wt % to about 85 wt/wt % of the one or more fillers.

35. The composition of claim 33 or 34, wherein the composition comprises from about 30 phr to about 500 phr of one or more fillers.

36. The composition of any one of claims 33-35, wherein the one or more fillers comprise carbon black, silica, kaolin, calcium silicate, talc, carbon nanotubes (CNT), carbon fibers (HCF), graphite, graphenes, aluminosilicates, starch, fibers, or a combination thereof.

37. The composition of claim 36, wherein the one or more fillers comprise silica.

38. The composition of claim 37, wherein the silica is derived from rice husks.

39. The composition of any one of claims 33-38, wherein the one or more fillers comprise carbon black.

40. The composition of any one of claims 1-39, wherein the composition comprises one or more rubber chemicals.

41. The composition of claim 40, wherein the composition comprises from about 15 wt/wt % to about 85 wt/wt % of the compound and from about 15 wt/wt % to about 85 wt/wt % of one or more rubber chemicals.

42. The composition of claims 40 or 41, wherein the composition comprises from about 0.1 phr to about 30 phr of one or more rubber chemicals.

43. The composition of claim 42, wherein the composition comprises from about 1 phr to about 20 phr of one or more rubber chemicals.

44. The composition of any one of claims 40-43, wherein the one or more rubber chemicals comprise one or more vulcanizing agents, one or more accelerators, one or more activators, one or more pre-vulcanization inhibitors, or a combination thereof.

45. The composition of claim 44, wherein the one or more rubber chemicals comprise one or more vulcanizing agents.

46. The composition of claim 45, wherein the one or more vulcanizing agents comprise sulfur, peroxide, resin, or a combination thereof.

47. The composition of claim 46, wherein the sulfur is octasulfur (S₈), cyclododecasulfur (S₁₂), polymeric sulfur, or a combination thereof.

48. The composition of claim 46, wherein the peroxide is benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy)hexane (DDPH), di-(2-tert-butylperoxyisopropyl)benzene (VC), butyl-4,4-di-(tert-butylperoxy)valerate (VAL), 1,1-di(tert-butylperoxy)-3,3,5-trimethylcyclohexane (TMC), or a combination thereof.

49. The composition of claim 46, wherein the resin is a bonding resin.

50. The composition of claims 44-49, wherein the one or more rubber chemicals comprise one or more accelerators.

51. The composition of claim 50, wherein the one or more accelerators comprise a guanidine, a thiazole, a sulfenamide, a sulfenamide, a thiuram, a dithiocarbamate, a xanthate, a thiophosphate, or a combination thereof.

52. The composition of claim 51, wherein the guanidine is diphenylguanidine (DPG).

53. The composition of claim 51, wherein the thiazole comprises 2-mercaptobenzothiazole (MBT), zinc 2-mercaptobenzothiazole (ZMBT), mercaptobenzothiazole disulfide (MBTS), N-tert-butyl-2-benzothiazole sulfenamide (TBSI), or a combination thereof.

54. The composition of claim 51, wherein the sulfenamide comprises N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), thiocarbamyl sulfenamide, or a combination thereof.

55. The composition of claim 51, wherein the thiuram is dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), tetramethylthiuram monosulfide (TMTM), or a combination thereof.

56. The composition of claim 51, wherein the dithiocarbamate comprises zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), zinc dibenzyldithiocarbamate (ZEBC), or a combination thereof.

57. The composition of any one of claims 44-56, wherein the one or more rubber chemicals comprise one or more activators.

58. The composition of claim 57, wherein the one or more activators comprise a metal oxide, an acid, a metal complex, or a combination thereof.

59. The composition of claim 58, wherein the metal oxide comprises zinc oxide, magnesium oxide, lead oxide, or a combination thereof.

60. The composition of claim 58, wherein the acid comprises stearic acid, lauric acid, or a combination thereof.

61. The composition of claim 58, wherein the metal complex comprises zinc ethylhexanoate.

62. The composition of any one of claims 44-61, wherein the one or more rubber chemicals comprise one or more pre-vulcanization inhibitors.

63. The composition of claim 62, wherein the one or more pre-vulcanization inhibitors comprise N-(cyclohexylthio)phthalimide (CTP), benzoic anhydride, salicylic anhydride, phthalic anhydride, or a combination thereof.

64. The composition of any one of claims 1-63, wherein the composition comprises one or more plasticizers.

65. The composition of claim 64, wherein the composition comprises from about 15 wt/wt % to about 85 wt/wt % of the compound and from about 15 wt/wt % to about 85 wt/wt % of one or more plasticizers.

66. The composition of claim 64 or 65, wherein the composition comprises from about 0.1 phr to about 30 phr of one or more plasticizers.

67. The composition of claim 66, wherein the composition comprises from about 1 phr to about 20 phr of one or more plasticizers.

68. The composition of any one of claims 64-67, wherein the one or more plasticizers comprise a mineral oil, an organic ester, a resin, a wax, an ester plasticizer, a naturally derived oil, or a combination thereof.

69. The composition of claim 68, wherein the mineral oil is naphthenic oil, paraffinic oil, or aromatic oil.

70. The composition of claim 68, wherein the naturally derived oil is soybean oil, vegetable oil, or orange oil.

71. The composition of any one of claims 1-70, wherein the composition further comprises a second antidegradant.

72. The composition of claim 71, wherein the composition comprises from about 15 wt/wt % to about 85 wt/wt % of the compound and from about 15 wt/wt % to about 85 wt/wt % of the second antidegradant.

73. The composition of claim 71 or 72, wherein the second antidegradant is present in an amount of from about 0.001 phr to about 10 phr.

74. The composition of claim 73, wherein the second antidegradant is present in an amount of from about 0.1 phr to about 5 phr.

75. The composition of claim 74, wherein the second antidegradant is present in an amount of from about 0.5 phr to about 5 phr.

76. The composition of claim 75, wherein the second antidegradant is present in an amount of from about 1 phr to about 5 phr.

77. The composition of any one of claims 71-76, wherein the second antidegradant is an antioxidant.

78. The composition of any one of claims 71-76, wherein the second antidegradant is an antiozonant.

79. The composition of any one of claims 71-78, wherein the second antidegradant is a paraphenylenediamine (PPD), a trimethyldihydroquinoline (TMQ), a phenolic, an alkylated diphenylamine (DPA), or a diphenylamine-ketone condensate.

80. The composition of claim 79, wherein the PPD is N¹-(4-methylpentan-2-yl)-N⁴-phenylbenzene-1,4-diamine (6PPD), N-(1,4-dimethylpentyl)-N′-phenyl-p-phenylenediamine (7PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), or N—N′-dioctyl-p-phenylenediamine (88PD).

81. The composition of claim 80, wherein the PPD is 6PPD.

82. The composition of claim 79, wherein the TMQ is 2,2,4-trimethyl-1,2-dihydroquinoline or an oligomer or polymer thereof.

83. A composition comprising a compound having Formula (I): 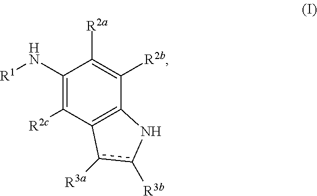
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{3c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl; and one or more carriers.

84. The composition of claim 83, wherein [symbol] is a double bond.

85. The composition of claim 83, wherein [symbol] is a single bond.

86. The composition of any one of claims 83-85, wherein R¹ is selected from the group consisting of C₁-C₁₂ alkyl and optionally substituted phenyl.

87. The composition of any one of claims 83-86, wherein R¹ is phenyl.

88. The composition of any one of claims 83-86, wherein R¹ is selected from the group consisting of methyl, ethyl, propyl, isopropyl, tert-butyl, sec-butyl, iso-butyl, pentyl, hexyl, heptyl, octyl, nonyl, decyl, undecyl, and dodecyl.

89. The composition of claim 88, wherein R¹ is sec-butyl.

90. The composition of claim 88, wherein R¹ is isopropyl.

91. The composition of any one of claims 83-85, wherein:
R¹ is —CHR^{1a}R^{1b};
R^{1a} is selected from the group consisting of: 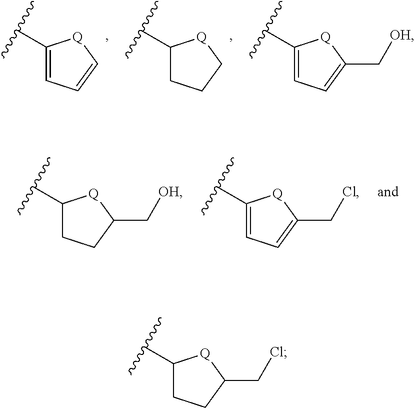
Q is selected from the group consisting of —NH—, —O—, and —S—; and
R^{1b} is selected from the group consisting of hydrogen and methyl.

92. The composition of any one of claims 83-91, wherein R^{2a}, R^{2b}, and R^{2c} are hydrogen.

93. The composition of any one of claims 83-94, wherein R^{3a} and R^{3b} are hydrogen.

94. The composition of claim 83, wherein the compound is selected from any one or more of the compounds of Table 1.

95. A process for preparing the composition of any one of claims 83-94, the process comprising admixing the compound and the one or more carriers.

96. A vulcanized elastomeric article comprising a compound having Formula (I): 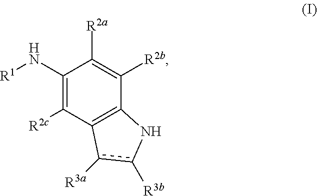
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{3c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl; and one or more carriers.

97. The article of claim 96, wherein [symbol] is a double bond.

98. The article of claim 96, wherein [symbol] is a single bond.

99. The article of any one of claims 96-98, wherein R¹ is selected from the group consisting of C₁-C₁₂ alkyl and optionally substituted phenyl.

100. The article of any one of claims 96-98, wherein R¹ is phenyl.

101. The article of any one of claims 96-98, wherein R¹ is selected from the group consisting of methyl, ethyl, propyl, isopropyl, tert-butyl, sec-butyl, iso-butyl, pentyl, hexyl, heptyl, octyl, nonyl, decyl, undecyl, and dodecyl.

102. The article of claim 101, wherein R¹ is sec-butyl.

103. The article of claim 101, wherein R¹ is isopropyl.

104. The article of any one of claims 96-98, wherein:
R¹ is —CHR^{1a}R^{1b};
R^{1a} is selected from the group consisting of: 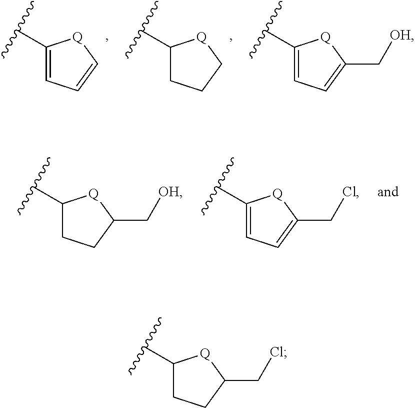
Q is selected from the group consisting of —NH—, —O—, and —S—; and
R^{1b} is selected from the group consisting of hydrogen and methyl.

105. The article of any one of claims 96-104, wherein R^{2a}, R^{2b}, and R^{2c} are hydrogen.

106. The article of any one of claims 96-104, wherein R^{3a} and R^{3b} are hydrogen.

107. The article of claim 96, wherein the compound is selected from any one or more of the compounds of Table 1.

108. A vulcanized elastomeric article comprising the composition of any one of claims 1-94.

109. A vulcanized elastomeric article prepared using the composition of any one of claims 1-94.

110. The vulcanized elastomeric article of claims 108 or 109, wherein the vulcanized elastomeric article is a tire.

111. The vulcanized elastomeric article of claim 110, wherein the tire is a passenger vehicle tire, a light truck tire, a heavy truck or bus tire, a motorcycle tire, an agriculture tire, an earthmover tire, an airplane tire, or a racing tire.

112. The vulcanized elastomeric article of claims 109 or 109, wherein the vulcanized elastomeric article is a component of a tire.

113. The vulcanized elastomeric article of claim 112, wherein the component is a bead, a belt, a body ply, an inner liner, a sidewall, an undertread, or a tread.

114. The vulcanized elastomeric article of claims 108 or 109, wherein the vulcanized elastomeric article is a rubber overshoe, a sealing strip, an acoustic panel, an air spring, a bellow, a membrane, a tactile sensor, a crash pad, a hose, a conveyor belt, or a flooring.

115. A process for preparing a vulcanized elastomeric article, the process comprising:
(a) forming the composition of any one of claims 13-82 into a formed shape; and
(b) vulcanizing the formed shape
to provide a vulcanized elastomeric article.

116. The process of claim 115, wherein the vulcanizing is performed at an average temperature of from about 120° C. to about 180° C.

117. The process of claim 116, wherein the vulcanizing is performed at an average temperature of from about 140° C. to about 160° C.

118. The process of any one of claims 115-117, wherein the vulcanized elastomeric article is a tire.

119. The process of claim 118, wherein the tire is a passenger vehicle tire, a light truck tire, a heavy truck or bus tire, a motorcycle tire, an agriculture tire, an earthmover tire, an airplane tire, or a racing tire.

120. The process of any one of claims 115-117, wherein the vulcanized elastomeric article is a component of a tire.

121. The process of claim 120, wherein the component is a bead, a belt, a body ply, an inner liner, a sidewall, an undertread, or a tread.

122. The process of any one of claims 115-121, wherein the vulcanized elastomeric article is a rubber overshoe, a sealing strip, an acoustic panel, an air spring, a bellow, a membrane, a tactile sensor, a crash pad, a hose, a conveyor belt, or a flooring.

123. A lubricant composition comprising a lubricant and a compound having Formula (I): 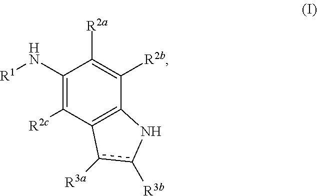
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{3c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl.

124. A combustible fuel composition comprising a combustible fuel and a compound having Formula (I): 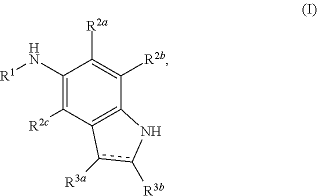
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{3c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl.

125. A fuel additive composition comprising a fuel additive and a compound having Formula (I): 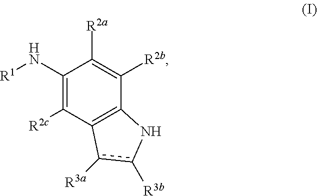
or a salt or solvate thereof, wherein: [symbol]
is a single or a double bond;
R¹ is selected from the group consisting of C₁-C₁₂ alkyl, —CHR^{1a}R^{1b}, optionally substituted C₃-C₆ cycloalkyl, optionally substituted 4- to 6-membered heterocyclyl, optionally substituted phenyl, and optionally substituted 5- or 6-membered heteroaryl;
R^{1a} is selected from the group consisting of optionally substituted phenyl, optionally substituted C₃-C₆ cycloalkyl, optionally substituted 4- to 6-membered heterocyclyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, 4- to 6-membered heterocyclyl, optionally substituted phenyl, and optionally substituted 5- or 6-membered heteroaryl; or
R^{2a} and R^{2b} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{3c} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl;
R^{3a} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, optionally substituted phenyl, and optionally substituted 5- or 6-membered heteroaryl; and
R^{3b} is selected from the group consisting of hydrogen, halogen, C₁-C₉ alkyl, C₁-C₉ haloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NR⁴R⁵, —SC(═O)R⁴, —SC(═O)SR⁴, —SC(═O)NR⁴R⁵, —NR⁴C(═O)SR⁵, —NR⁴C(═S)SR⁵, optionally substituted 4- to 6-membered heterocyclyl, and optionally substituted 5- or 6-membered heteroaryl; or
R^{3a} and R^{3b} taken together with the two carbon atoms to which they are attached form an optionally substituted C₃-C₁₂ cycloalkyl or an optionally substituted 4- to 6-membered heterocyclyl;
R⁴ is selected from the group consisting of hydrogen and C₁-C₉ alkyl; and
R⁵ is selected from the group consisting of hydrogen and C₁-C₉ alkyl.

126. A process for retreading tires, the process comprising:
(a) applying the composition of any one of claims 13-82 to a tire;
(b) disposing a pre-vulcanized tread around the tire;
(c) disposing a curing envelope around the tire; and
(d) vulcanizing the tire.

127. A kit comprising the composition of any one of claims 1-94 and instructions for using the composition in a vulcanizable elastomeric composition.

128. A kit comprising the composition of any one of claims 1-94 and instructions for using the composition to prepare a vulcanized elastomeric article.

129. The composition of any one of claims 1-12, wherein the composition comprises from about 0.1 wt/wt % to about 10 wt/wt % of the compound.

130. The composition of any one of claims 83-94, wherein the composition comprises from about 0.1 wt/wt % to about 10 wt/wt % of the compound.
